SEQUENCE LISTING (5 sequences)

SEQ ID NO: 1 - protein, 805 aa, Homo sapiens [sequence omitted]

SEQ ID NO: 2 - protein, 723 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 3 - protein, 723 aa, Homo sapiens
QSTIEEQAKTFLDKFNHEAEDLFYQSSLASWNYNTNITEENVQNMNNAGDKWSAFLKEQSTLAQMYPLQEIQDAKIKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 4 - protein, 463 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEM

SEQ ID NO: 5 - protein, 463 aa, Homo sapiens
QSTIEEQAKTFLDKFNHEAEDLFYQSSLASWNYNTNITEENVQNMNNAGDKWSAFLKEQSTLAQMYPLQEIQDAKIKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEM

(19) United States
(12) Patent Application Publication
(10) Pub. No.: US 2023/0414726 A1
(43) Pub. Date: Dec. 28, 2023

(54) RESPIRABLE AQUEOUS PHARMACEUTICAL COMPOSITION COMPRISING A POLYPEPTIDE FOR CORONA VIRUS TREATMENT AND NEUTRALIZATION

(71) Applicant: MASKER MED TECH AB, Malmö (SE)

(72) Inventors: György MARKO-VARGA, Malmö (SE); Yutaka SUGIHARA, Malmö (SE); Jeovanis GIL VALDÉS, Lund (SE); Roger APPELQVIST, Malmö (SE); Johan MALM, Malmö (SE)

(21) Appl. No.: 18/035,458

(22) PCT Filed: Nov. 9, 2021
(86) PCT No.: PCT/SE2021/051122

§ 371 Date: May 4, 2023

(30) Foreign Application Priority Data
Nov. 9, 2020 (SE) 2051304-0

(51) Int. Cl. A61K 38/48; A61K 9/00; A61P 31/14; A61K 47/18; A61K 47/26; A61K 47/02; A61K 47/12

(52) U.S. Cl. CPC A61K 38/48; A61K 9/0078; C12Y 304/17023; A61P 31/14; A61K 47/183; A61K 47/26; A61K 47/02; A61K 47/12

(57) ABSTRACT

A respirable aqueous pharmaceutical composition comprising a neutralizing affinity binder for a virus binding to angiotensin-converting enzyme 2 (ACE2), a buffer, and a solubilizer.

### FIELD OF THE INVENTION

This invention pertains in general to a respirable aqueous pharmaceutical composition comprising a neutralizing affinity binder, a buffer and a solubilizer. More particularly the invention relates a solution that enables use of cell surface receptor angiotensin-converting enzyme 2 (ACE2), or domains thereof, for virus neutralization or treatment. More particularly, the invention relates to a solution for use in the neutralization or treatment of viruses binding to ACE2.

### BACKGROUND OF THE INVENTION

Coronavirus disease 2019 (COVID-19) caused by the novel COVID-19 virus has turned into a pandemic. The virus has spread worldwide, causing fever, severe respiratory illness, and pneumonia (C. Wang et al. Lancet 395, 470-473 (2020) and N. Zhu, et al. 2019. N. Engl. J. Med. 382, 727-733 (2020)). By phylogenetic analysis there is a clear indication that the virus is closely related to severe acute respiratory syndrome coronavirus (SARS-CoV) (P. Zhou, A et al., Nature 579, 270-273 (2020) and R. Lu, et al., Lancet 395, 565-574 (2020)), however, it appears to be more easily transmitted from person to person than SARS-CoV (J. Chan, et al., Lancet 395, 514-523 (2020)). To date, no specific drugs or vaccines are available for COVID-19.

By now its manifested that the COVID-19 virus belongs to the betacoronavirus genus, that includes five pathogens which infect humans. Amongst these, severe acute respiratory syndrome coronavirus (SARS-CoV) and Middle East respiratory syndrome coronavirus (MERS-CoV) are two of the most well-known pathogenic viruses. As with other coronaviruses, the spike (S) glycoprotein homotrimer on the COVID-19 virus surface plays an essential role in receptor binding and virus entry. It is known by now that the S protein is a class I fusion protein, where each S protomer consists of S1 and S2 domains. This, in an affinity interaction with the receptor binding domain (RBD) located within the S1 domain. Previous studies have revealed that the COVID-19 virus, similarly to SARS-CoV, uses the angiotensin-converting enzyme 2 (ACE2) receptor for cell entry. Another Human coronavirus using ACE2 for receptor for cell entry is NL63 (HCoV-NL63), which is a species of coronavirus primarily found in young children, the elderly, and immunocompromised patients with acute respiratory illness.

Currently, specific and effective drugs are unavailable and the mainstay of COVID-19 treatment is supportive care. At the same time, enormous efforts are currently carried out globally to identify drugs that may be effective against SARS-CoV-2 infection. These approaches are typically based on the idea of drug repurposing (e.g. using the antimalarial drug chloroquine or antiviral agents (e.g. favipiravir) not designed directly against coronaviruses).

It is clear there is a need for any treatment approaches that are specifically targeted against coronaviruses, not only for known pathogens like SARS-CoV-2, but also for future viruses using the ACE2 receptor for cell entry.

### SUMMARY OF THE INVENTION

Accordingly, the present invention preferably seeks to mitigate, alleviate or eliminate one or more of the above-identified deficiencies in the art and disadvantages singly or in any combination and solves at least the above mentioned problems by providing a respirable aqueous pharmaceutical composition comprising a neutralizing affinity binder for viruses binding to angiotensin-converting enzyme 2 (ACE2), a buffer and a solubilizer.

### BRIEF DESCRIPTION OF THE DRAWINGS

These and other aspects, features and advantages of which the invention is capable of will be apparent and elucidated from the following description of embodiments of the present invention, reference being made to the accompanying drawings, in which

FIG. 1 shows the mechanism of SARS-CoV-2 entry into the cells of the bronchial/alveolar wall and proposed therapeutic intervention. FIG. 1.A. SARS-CoV-2 infection is mediated by the interaction of the viral spike (S) protein and its functional receptor, ACE2. The plasma membrane forms an endosome around the virus and the virus enters cells by endocytosis. FIG. 1. B. Inhalation of soluble ACE2 saturates the available S proteins, blocks the abovementioned interaction and abrogates infection;

FIG. 2 shows the sequence coverage confirmation of peptide 2MA1-main band, by mass spectrometry, the bands representing the fragments sequenced;

FIG. 3 shows an MS/MS spectrum of the double charged signal with 517.26 as the molecular mass (the spectrum was matched with the theoretical sequence of the N-terminal peptide of 2MA1);

FIG. 4 shows MS/MS spectra of the triple charged signal 778.74 Th corresponding to the peptide from 2MA1 58-77 (A) and the double charged signal 649.32 Th from the peptide 325-336 (B);

FIG. 5 shows gel results for the stability of peptide 2MA1 which was investigated with perspective to pH-window and sample composition;

FIG. 6 shows gel results for the stability of ACE2 that was investigated by different buffer conditions (with pH 5 and 11);

FIG. 7 shows a plot of the binding properties of peptide 2MA1, to the SPIKE protein of SARS COVID-2;

FIG. 8 shows a plot of the peptide 2MA1 binding kinetics to RBD (receptor binding domain) of the SPIKE protein;

FIG. 9 shows a plot of de-glycosylated peptide 2MA1 binding kinetics to RBD of the SPIKE protein;

FIG. 10 shows gel results for binding of ECD (extra cellular domain of spike protein) to E. coli expressed ACE2 extracellular domain after 10 minutes incubation time; and

FIG. 11 shows gel results for binding of ECD to E. coli expressed ACE2 extracellular domain after 10 hours incubation time;

FIG. 12 shows mesh vaporization of the composition of the invention; and

FIG. 13 shows jet vaporization of the composition of the invention.

FIG. 14 shows MS/MS spectra of the quadruple charged signal 1077.97 Th corresponding to the peptide from 2MA1 15-51 highlighting the glycosylation site at position N36 (A), the double charged signal 1168.09 Th corresponding to the peptide 58-77 glycosylated at N73 (B) and the double charged signal corresponding to the peptide 78-95 from 2MA1 glycosylated in N86 (C);

FIG. 15 shows intrapulmonary levels of ACE2 (2MA1) determined by Western blot analysis after injecting 1 μg protein/mouse;

FIG. 16 shows intrapulmonary levels of ACE2 (2MA1) determined by Western blot analysis after injecting 5 μg protein/mouse;

FIG. 17. shows plasma levels of ACE2 (2MA1) after intrapulmonary delivery of the protein;

FIG. 18. shows representative histology images of the lungs of control mice 30 min/6 h/24 h/48 h after injection;

FIG. 19. shows representative histology images of the lungs of mice that received 1 μg ACE2 (2MA1) 30 min/6 h/24 h/48 h after injection;

FIG. 20. shows representative histology images of the lungs of mice that received 5 μg ACE2 (2MA1) 30 min/6 h/24 h/48 h after injection;

FIG. 21 shows the RBDDB (here SARS-CoV-2 Spike S1 Receptor Binding Domain Protein) and 2MA1 kinetics in mouse lung;

FIG. 22 shows gel results for filtration procedure for, wherein the first two columns show that free RBD (here SARS-CoV-2 Spike S1 Receptor Binding Domain Protein) go into the flow-through fraction, while the second two columns shows that the RDBRBD-ACE2 protein complex stayed in the filtration device;

FIG. 23 shows identified 2MA1 protein sequence by MS analysis in mouse lung experiments;

FIG. 24 shows identified RDBRBD (here SARS-CoV-2 Spike S1 Receptor Binding Domain Protein) protein sequence by MS analysis in mouse lung experiments;

FIG. 25 shows mass spectra correctly assigned to peptide sequences from 2MA1 marked in FIG. 23;

FIG. 26 shows mass spectra correctly assigned to peptide sequences from 2MA1 marked in FIG. 23;

FIG. 27 shows mass spectra correctly assigned to peptide sequences from RBD protein sequence marked in FIG. 24;

FIG. 28 shows mass spectra correctly assigned to peptide sequences from RBD protein sequence marked in FIG. 24;

FIG. 29 shows MS/MS spectra with a comparison of signals between the supernatant (top) and the flow-through (bottom) in mouse lung experiments; and

FIG. 30 shows MS/MS spectra with a comparison of signals between the supernatant (top) and the flow-through (bottom) in mouse lung experiments.

### DESCRIPTION OF EMBODIMENTS

The following description focuses on an embodiment of the present invention applicable to a respirable aqueous pharmaceutical composition comprising a neutralizing affinity binder for viruses binding to angiotensin-converting enzyme 2 (ACE2), a buffer and a solubilizer wherein the neutralizing affinity binder is a polypeptide comprising the sequence of cell surface receptor angiotensin-converting enzyme 2 (ACE2), or part of the sequence. In particular, a polypeptide according to the invention, such as SEQ ID NO 2 or SEQ ID NO 3.

The recent Coronavirus pandemics, SARS and COVID-19, caused by SARS-CoV and SARS-CoV-2, respectively, utilize ACE2 as a receptor to enter the target cell. However, also other virus infections, such as HCoV-NL63 (Human coronavirus NL63), have been attributed to entering its host cell by binding to ACE2. It has been speculated that the soluble form of ACE2 may compete with the membrane-bound form and thus inhibiting viral infection. Indeed, ACE2 expression on different cell lines correlates with susceptibility to SARS-CoV infection.

Although ACE2 is expressed in various organs (e.g. the kidneys and the gastrointestinal tract), type 2 pneumocytes express high amounts of ACE2. The extracellular domain of the full-length ACE2 is anchored to the plasma membrane by its transmembrane domain.

In a very recent report, clinical-grade recombinant human (rh) soluble ACE2 (hrsACE2) has been shown to significantly reduce SARS-CoV-2 viral growth in vitro. In line with this, a randomized, double-blind Phase II trial has been started to evaluate the efficacy and safety of the intravenously (i.v.) given recombinant form of ACE2 (APN01) in severely infected COVID-19 patients in Europe.

In the invention, we find that the optimum route of administration of ACE2, or a peptide according to the invention, is pulmonary inhalation. Not only does the lung offer a large surface area for drug absorption, importantly, the ACE2, or peptide of the invention, will act as a neutralizer for virus particles both in the upper and lower respiratory tract, and polypeptides and deactivated virus particles may both be expelled as phlegm, making administration safe for the COVID-19 patients.

When referring to ACE2 (for inhalation) below, it is understood that this refers to human ACE2 protein, or a peptide of the invention.

When referring to 2MA1 below, it is understood that this refers to a peptide of the invention with a sequence of SEQ ID NO 2, or a polypeptide comprises at least 700 amino acids, such as at least 710, such as at least 715, such as at least 718, 719, 720, 721, 722, 723 AA and having an amino acid sequence of at least 90%, such as at least 95%, 96%, 97, 98%, or 99%, such as 100% sequence identity (% SI) with SEQ ID NO 2 or SEQ ID NO 3.

Once locally administered, SARS-CoV-2 virus particles will bind to the ACE2 proteins (or peptide of the invention), either the administered (exogenous) ACE2, or on the host RCE2. Published surface plasmon resonance experiments probing the binding kinetics for human ACE2 and immobilized 2019-nCoV shows that SARS-CoV-2 S protein binds to the PD of ACE2 at high affinity (a dissociation constant (Kd) of ˜15 nM). Binding for the formulation comprising peptide 2MA1 of the invention can be seen in FIG. 8. As such, the administered ACE2, or peptide of the invention, will serve to occupy active virus particles, by competitive binding, thus neutralizing part of the virus particles.

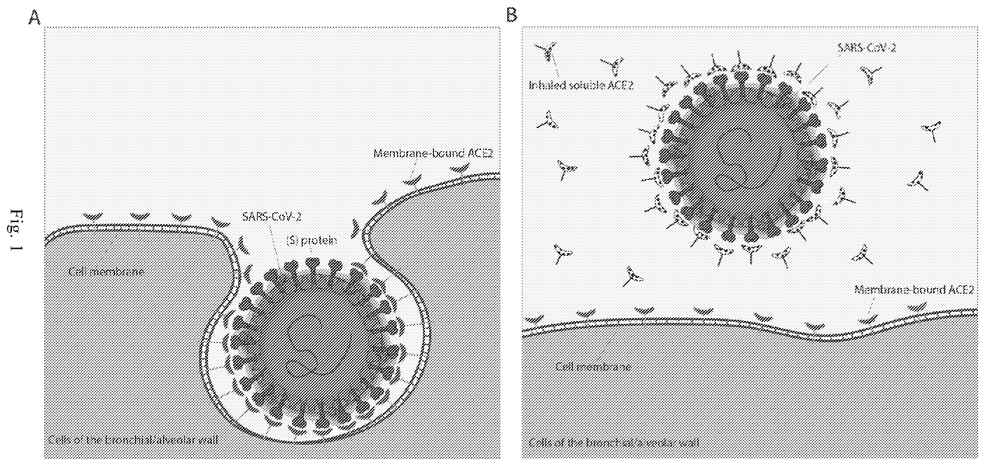

By introducing inhaled rhACE2, or a peptide according to the invention, into the COVID19-infected respiratory tract, a competitive action will take place with a dynamic equilibrium that will determine the affinity and binding kinetics of the virus particles for their receptors (i.e. host ACE2 vs. exogenous rhACE2). With a given dosing, the kinetic rate constants and equilibrium constants will favour the COVID19-rhACE2 complex formation. Similarly, by increasing the dose, the equilibrium can be pushed further towards COVID19-rhACE2 complex formation. This is effectually illustrated by FIG. 1, which shows an example of such virus particle neutralization.

In contrast to i.v. administration, inhalation is a promising non-invasive method of rhACE2 delivery to treat COVID19 patients, as it will result high drug levels in the lung, while, depending on drug formulation, limiting rhACE2 passage into the pulmonary capillaries (i.e. the circulation). Importantly, inhaled ACE2, or the peptide of the invention, will also avoid any first-pass metabolism in the liver.

Importantly, inhaled ACE2 (or peptide of the invention) can be used in COVID19 patients with less severe symptoms to reduce the number of virus particles in their exhaled breath and in this wise reduce their capability to infect other subjects.

Early data from COVID-19 patients suggest that a large amount of virus particles is present in patients' nasal cavities, possibly before they have symptoms and likely in the first week of the disease. As such, it is advantageous to use the peptide of the invention such that the peptide passes through the nasal cavity of the patient. This may happen automatically during pulmonary administration, for instance when administering an aerosol using a mask covering both mouth and nose of the patient. However, the nasal cavity may also be reached using simpler targeted administration, such as using a nose spray comprising the peptide of the invention, or by administering the peptide of the invention using any other suitable intranasal drug delivery system.

Thus, the composition of the invention can be used for neutralizing virus particles binding to ACE2.

Since ACE2 or the peptide of the invention (such as SEQ ID NO 2) is a large protein domain with a complicated fold, it is vulnerable to physical instability, such as high shear stresses during vaporization. Physical instability is usually referred to changes in the higher order structure of the biomolecule, such as the protein fold, usually without breakage of covalent bonds. Such forms of physical instability include the formation of dimers or larger aggregates or precipitation.

The three-dimensional structure of the peptides of the invention is key to the binding to the virus, why the fold has got to be protected and maintained during the stresses that are involved in the conversion to an aerosol suitable for inhalation.

In the invention, it was found that under certain conditions, it is indeed possible to maintain the delicate structure to have binding even after stress. This is extra important, since the protein will go from solution to vaporized droplets to the local environment of the nasal cavity or lung, all while maintaining the correct structure.

By finding the correct solution pH, buffer type, salt concentration, the net electrostatic surface repulsion of the structure may be managed, thus shielding the surface charge of the protein. This is especially important for an aerosol, where the solution will form micro droplets where air/water interfaces will be created, leading to interfacial stress. Furthermore, during formation of the aerosol, the protein will also be exposed to shear and cavitation stress.

Since the volume of the micro droplets of the aerosol will be very small in relation to the droplet surface area, the droplets will be sensitive to the micro environment humidity and temperature, making the protein in the droplet in risk of potential concentration-, ionic strength- and thermal stress.

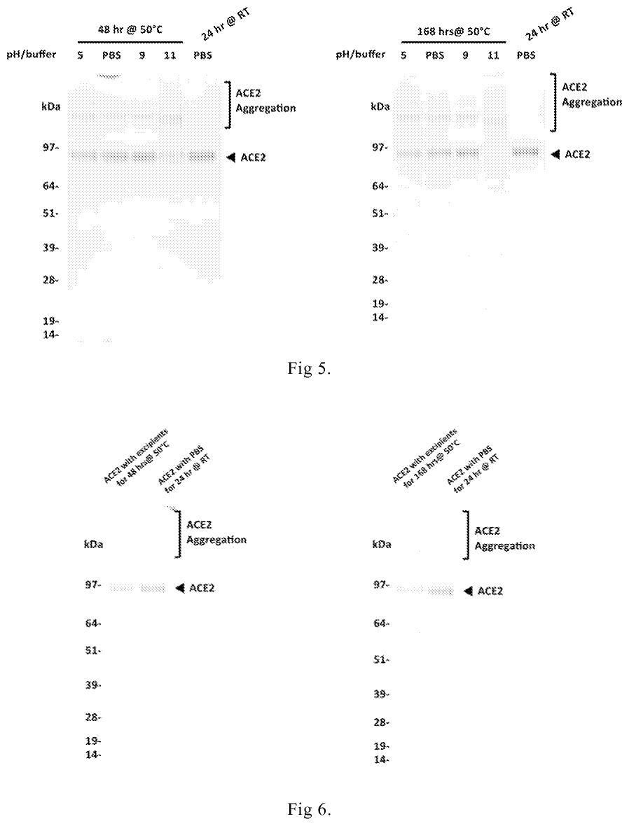

Thermal stress analysis was used to find a formulation where the peptide structure is maintained, ensuring to have binding even after thermal stress. As can be seen in FIG. 5, and it is clear that buffer and pH is important for peptide stability. As can be seen, the formulation of the invention does not aggregate but mainly remains in monomer form, even after 168 hours at degrees. It was also shown that the optimum pH for stability is 5 to 9.5, such as 6 to 8.

In one embodiment, the peptide is in monomer form.

In one embodiment, the composition has a pH of 4 to 10, preferably 5 to 9, more preferably 6 to 8.

In one embodiment, the composition has a pH of 5 to 9.5, preferably 6 to 8. The importance of suitable buffer was also shown. It was found that suitable buffers for stability within the desired pH range was citrate buffer, phosphate buffer, phosphate-buffered saline (PBS), MOPS (3-(N-morpholino)propanesulfonic)acid) buffer, HEPES (4-(2-hydroxyethyl)-1-piperazineethanesulfonic acid) buffer, DIPSO (3-(N,N-Bis[2-hydroxyethyl]amino)-2-hydroxypropanesulfonic acid) buffer, Tris-HCl buffer, or a combination of these.

In one embodiment, the composition comprises citrate buffer, phosphate buffer, phosphate-buffered saline (PBS), MOPS (3-(N-morpholino)propanesulfonic)acid) buffer, HEPES (4-(2-hydroxyethyl)-1-piperazineethanesulfonic acid) buffer, DIPSO (3-(N,N-Bis[2-hydroxyethyl]amino)-2-hydroxypropanesulfonic acid) buffer, Tris-HCl buffer or a combination of these. The buffer concentration may be at least 10 mM, such as at least 20 mM, such as from to 250 mM, such as from 20 to 250 mM, such as from 50 to 200 mM, such as 50 to 100 mM. In one embodiment, the composition comprises phosphate buffer, PBS buffer, or Tris-HCl buffer or a combination of these. In one embodiment, the composition comprises phosphate buffer.

Furthermore, it was found that the physical stability of the protein structure could be protected using a solubilizer. It was found that a non-ionic solubilizer such as a non-ionic solubilizers derived from sorbitan esters worked well for this purpose.

Examples of such solubilizer are Polysorbate 20, Tween 40, Tween 60, Tween 80, and Tween 85. Tween 80 (Polysorbat 80, C₆₄H₁₂₄O₂₆, polyoxyethylene sorbitan monooleate) improved both thermal stress resistance and shear and cavitation stress resistance. Tween 80 has a longer aliphatic tail and therefore more lipophilic, why it may prevent dimerization or aggregation between protein structures under physical stress. This preserves the monomeric confirmation, preventing dimerization or multimers, which complicate delivery.

In one embodiment, the solution comprises a solubilizer. The solubilizer may be a non-ionic solubilizer, or a polymeric non-ionic solubilizer. The solubilizer may be selected from a group consisting of Polysorbate 20, Tween 40, Tween 60, and Tween 80. These may also be named Polysorbate 20 (polyoxyethylene (20) sorbitan monolaurate), Polysorbate 40 (polyoxyethylene (20) sorbitan monopalmitate), Polysorbate 60 (polyoxyethylene (20) sorbitan monostearate), Polysorbate 80 (Polyoxyethylene (20) sorbitan monooleate), respectively. The number (20) following the ‘polyoxyethylene’ part refers to the total number of oxyethylene —(CH 2 CH 2 O)— groups found in the molecule. The number following the ‘polysorbate’ part is related to the type of fatty acid associated with the polyoxyethylene sorbitan part of the molecule. Monolaurate is indicated by 20, monopalmitate is indicated by 40, monostearate by and monooleate by 80. The solubilizer may also be a poloxamine solubilizer or a polysaccharide solubilizer. The solubilizer concentration may be 0.01 to 0.8 mg/ml, such as 0.05 to 0.5 mg/ml, such as 0.1 to 0.4 ml/ml, such as 0.2 mg/ml. In one embodiment, the solubilizer is Tween 80.

The composition may also contain salts, such as sodium chloride (NaCl). The salt sodium chloride helps in keeping proteins soluble and to mimic physiological conditions. The NaCl concentration may be 0.05 to 30 mg/ml, such as 1 to 20 mg/ml, such as 5 to 15 ml/ml, such as 8.5 mg/ml.

Thus, in an embodiment of the invention is provided a respirable aqueous pharmaceutical composition comprising a neutralizing affinity binder for viruses binding to angiotensin-converting enzyme 2 (ACE2), a buffer and a solubilizer.

The virus binding to angiotensin-converting enzyme 2 (ACE2) may comprise a receptor-binding domain (RBD) that binds specifically to the angiotensin-converting enzyme 2 (ACE2) specific endogenous receptor sequence to gain entry into host cells. Also, the neutralizing affinity bay bind specifically to the receptor-binding domain (RBD) of the virus binding to angiotensin-converting enzyme 2 (ACE2). The virus binding to angiotensin-converting enzyme 2 (ACE2) may be Severe acute respiratory syndrome coronavirus 2 (SARS-CoV-2), severe acute respiratory syndrome coronavirus (SARS-CoV) or Human coronavirus NL63 (HCoV-NL63).

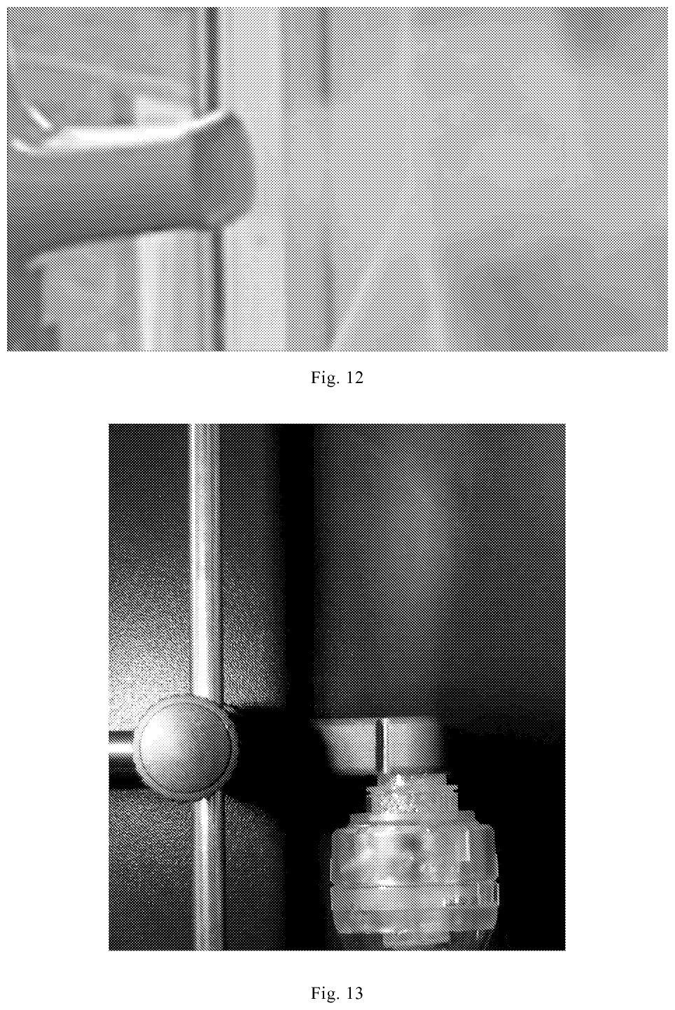

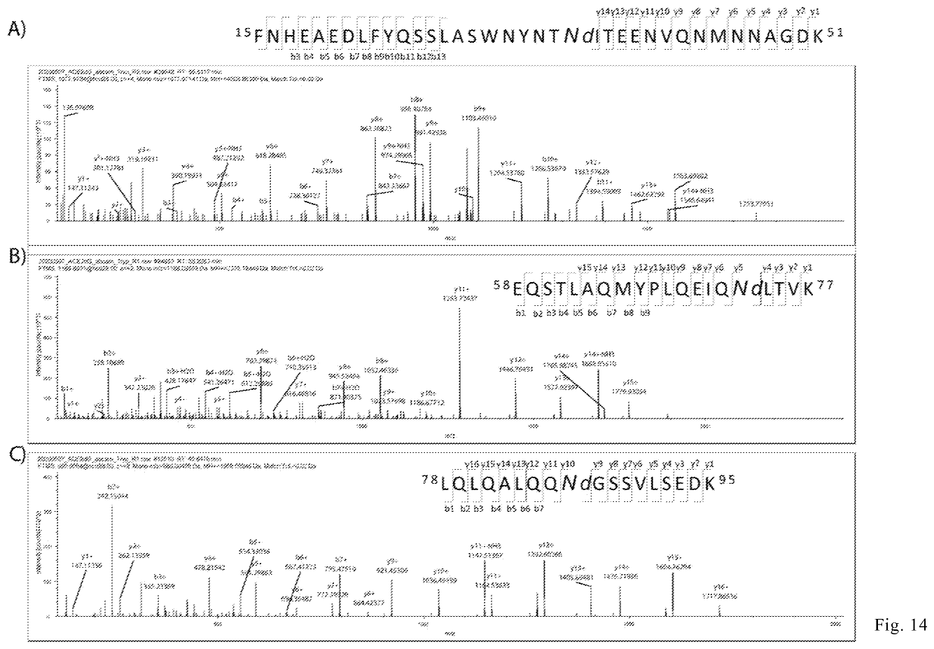

As can be seen in FIGS. 12 and 13, the respirable aqueous pharmaceutical composition of the invention works well with different types of dispensers, such as jet and mesh dispensers. The stickiness and viscosity enables both a low, medium and high dose of peptide to be vaporized, such as a peptide concentration of from 0.1 nM to 10 mM, such as 15, 30, 60, 125 and 250 nM peptide concentration (binding data for these can be seen in FIG. 8).

In one embodiment, the solution is made into an aerosol using a jet nebulizer or a mesh nebulizer.

It was also found that stability was improved by the addition of a chelating agent. Examples of chelating agents are Ethylenediaminetetraacetic acid (EDTA), nitriloacetic acid (NTA), phospohates, citric acid, and glycine. EDTA chelates the zinc ion required for metalloprotease activity which appears to improve structural stability during physical stress. Also, quenching the metalloprotease activity may be an advantage, since the peptide will not have several parallel effects, which may make it easier to evaluate treatment results.

In one embodiment, the solution comprises a chelating agent. The chelating agent may be selected from the group consisting of ethylenediaminetetraacetic acid (EDTA), nitriloacetic acid (NTA), phospohates, citric acid, and glycine. Preferably, the chelating agent is EDTA. Further, the chelating agent concentration may be 0.01 to 0.8 mg/ml, such as 0.05 to 0.5 mg/ml, such as 0.1 to 0.2 ml/ml, such as 0.1 mg/ml. In one embodiment, the chelating agent is ethylenediaminetetraacetic acid (EDTA).

Human ACE2 has the sequence according to Uniprot reference Q9BYFI (ACE2 HUMAN Angiotensin-converting enzyme), provided as SEQ ID NO 1, as shown in table 1.

TABLE 1
SEQUENCE LIST

SEQ ID NO 1: (Human ACE2),
MSSSSWLLLSLVAVTAAQSTIEEQAKTFLDKFNHEAEDLF
YQSSLASWNYNTNITEENVQNMNNAGDKWSAFLKEQSTLA
QMYPLQEIQNLTVKLQLQALQQNGSSVLSEDKSKRLNTIL
NTMSTIYSTGKVCNPDNPQECLLLEPGLNEIMANSLDYNE
RLWAWESWRSEVGKQLRPLYEEYVVLKNEMARANHYEDYG
DYWRGDYEVNGVDGYDYSRGQLIEDVEHTFEEIKPLYEHL
HAYVRAKLMNAYPSYISPIGCLPAHLLGDMWGRFWTNLYS
LTVPFGQKPNIDVTDAMVDQAWDAQRIFKEAEKFFVSVGL
PNMTQGFWENSMLTDPGNVQKAVCHPTAWDLGKGDFRILM
CTKVTMDDFLTAHHEMGHIQYDMAYAAQPFLLRNGANEGF
HEAVGEIMSLSAATPKHLKSIGLLSPDFQEDNETEINFLL
KQALTIVGTLPFTYMLEKWRWMVFKGEIPKDQWMKKWWEM
KREIVGVVEPVPHDETYCDPASLFHVSNDYSFIRYYTRTL
YQFQFQEALCQAAKHEGPLHKCDISNSTEAGQKLFNMLRL
GKSEPWTLALENVVGAKNMNVRPLLNYFEPLFTWLKDQNK
NSFVGWSTDWSPYADQSIKVRISLKSALGDKAYEWNDNEM
YLFRSSVAYAMRQYFLKVKNQMILFGEEDVRVANLKPRIS
FNFFVTAPKNVSDIIPRTEVEKAIRMSRSRINDAFRLNDN
SLEFLGIQPTLGPPNQPPVSIWLIVFGVVMGVIVVGIVIL
IFTGIRDRKKKNKARSGENPYASIDISKGENNPGFONTDD
VQTSF
SEQ ID NO 2
(extra cellular domain of the ACE2):
QSTIEEQAKTFLDKFNHEAEDLFYQSSLASWNYNTNITEE
NVQNMNNAGDKWSAFLKEQSTLAQMYPLQEIQNLTVKLQL
QALQQNGSSVLSEDKSKRLNTILNTMSTIYSTGKVCNPDN
PQECLLLEPGLNEIMANSLDYNERLWAWESWRSEVGKOLR
PLYEEYVVLKNEMARANHYEDYGDYWRGDYEVNGVDGYDY
SRGQLIEDVEHTFEEIKPLYEHLHAYVRAKLMNAYPSYIS
PIGCLPAHLLGDMWGRFWTNLYSLTVPFGQKPNIDVTDAM
VDQAWDAQRIFKEAEKFFVSVGLPNMTQGFWENSMLTDPG
NVQKAVCHPTAWDLGKGDFRILMCTKVTMDDFLTAHHEMG
HIQYDMAYAAQPFLLRNGANEGFHEAVGEIMSLSAATPKH
LKSIGLLSPDFQEDNETEINFLLKQALTIVGTLPFTYMLE
KWRWMVFKGEIPKDQWMKKWWEMKREIVGVVEPVPHDETY
CDPASLFHVSNDYSFIRYYTRTLYQFQFQEALCQAAKHEG
PLHKCDISNSTEAGQKLFNMLRLGKSEPWTLALENVVGAK
NMNVRPLLNYFEPLFTWLKDQNKNSFVGWSTDWSPYADQS
IKVRISLKSALGDKAYEWNDNEMYLFRSSVAYAMRQYFLK
VKNQMILFGEEDVRVANLKPRISFNFFVTAPKNVSDIIPR
TEVEKAIRMSRSRINDAFRLNDNSLEFLGIQPTLGPPNQP
PVS
SEQ ID NO 3
(extra cellular domain of the
ACE2 with mutation that altered the
NxT/S motif):
QSTIEEQAKTFLDKFNHEAEDLFYQSSLASWNYNTNITEE
NVQNMNNAGDKWSAFLKEQSTLAQMYPLQEIQDAKIKLQL
QALQQNGSSVLSEDKSKRLNTILNTMSTIYSTGKVCNPDN
PQECLLLEPGLNEIMANSLDYNERLWAWESWRSEVGKOLR
PLYEEYVVLKNEMARANHYEDYGDYWRGDYEVNGVDGYDY
SRGQLIEDVEHTFEEIKPLYEHLHAYVRAKLMNAYPSYIS
PIGCLPAHLLGDMWGRFWTNLYSLTVPFGQKPNIDVTDAM
VDQAWDAQRIFKEAEKFFVSVGLPNMTQGFWENSMLTDPG
NVQKAVCHPTAWDLGKGDFRILMCTKVTMDDFLTAHHEMG
HIQYDMAYAAQPFLLRNGANEGFHEAVGEIMSLSAATPKH
LKSIGLLSPDFQEDNETEINFLLKQALTIVGTLPFTYMLE
KWRWMVFKGEIPKDQWMKKWWEMKREIVGVVEPVPHDETY
CDPASLFHVSNDYSFIRYYTRTLYQFQFQEALCQAAKHEG
PLHKCDISNSTEAGQKLFNMLRLGKSEPWTLALENVVGAK
NMNVRPLLNYFEPLFTWLKDQNKNSFVGWSTDWSPYADQS
IKVRISLKSALGDKAYEWNDNEMYLFRSSVAYAMRQYFLK
VKNQMILFGEEDVRVANLKPRISFNFFVTAPKNVSDIIPR
TEVEKAIRMSRSRINDAFRLNDNSLEFLGIQPTLGPPNQP
PVS
SEQ ID NO 4
(ACE2 extracellular subdomain I):
QSTIEEQAKTFLDKFNHEAEDLFYQSSLASWNYNTNITEE
NVQNMNNAGDKWSAFLKEQSTLAQMYPLQEIQNLTVKLQL
QALQQNGSSVLSEDKSKRLNTILNTMSTIYSTGKVCNPDN
PQECLLLEPGLNEIMANSLDYNERLWAWESWRSEVGKOLR
PLYEEYVVLKNEMARANHYEDYGDYWRGDYEVNGVDGYDY
SRGQLIEDVEHTFEEIKPLYEHLHAYVRAKLMNAYPSYIS
PIGCLPAHLLGDMWGRFWTNLYSLTVPFGQKPNIDVTDAM
VDQAWDAQRIFKEAEKFFVSVGLPNMTQGFWENSMLTDPG
NVQKAVCHPTAWDLGKGDFRILMCTKVTMDDFLTAHHEMG
HIQYDMAYAAQPFLLRNGANEGFHEAVGEIMSLSAATPKH
LKSIGLLSPDFQEDNETEINFLLKQALTIVGTLPFTYMLE
KWRWMVFKGEIPKDQWMKKWWEM
SEQ ID NO 5
(ACE2 extracellular subdomain I
with mutation that altered the NxT/S
motif):
QSTIEEQAKTFLDKFNHEAEDLFYQSSLASWNYNTNITEE
NVQNMNNAGDKWSAFLKEQSTLAQMYPLQEIQDAKIKLQL
QALQQNGSSVLSEDKSKRLNTILNTMSTIYSTGKVCNPDN
PQECLLLEPGLNEIMANSLDYNERLWAWESWRSEVGKQLR
PLYEEYVVLKNEMARANHYEDYGDYWRGDYEVNGVDGYDY
SRGQLIEDVEHTFEEIKPLYEHLHAYVRAKLMNAYPSYIS
PIGCLPAHLLGDMWGRFWTNLYSLTVPFGQKPNIDVTDAM
VDQAWDAQRIFKEAEKFFVSVGLPNMTQGFWENSMLTDPG
NVQKAVCHPTAWDLGKGDFRILMCTKVTMDDFLTAHHEMG
HIQYDMAYAAQPFLLRNGANEGFHEAVGEIMSLSAATPKH
LKSIGLLSPDFQEDNETEINFLLKQALTIVGTLPFTYMLE
KWRWMVFKGEIPKDQWMKKWWEM

Importantly, SARS-CoV-2 spike protein does not bind to all of ACE2. The spike protein only interacts with part of the extracellular domain of the ACE2. The extracellular domain of ACE2 is amino acids 18 to 740 of SEQ ID NO 1 (1 to 17 is the signal peptide that might be removed upon activation, 741 to 761 is the transmembrane domain and 762 to 805 the cytoplasmic domain), which is shown as SEQ ID NO 2 in table 1, and is referred to as sequence 2MA1 herein.

In one embodiment is provided a respirable aqueous pharmaceutical composition comprising a polypeptide comprising at least 770 amino acids, such as at least 780, such as at least 790, such as at least 800, 801, 802, 803, 804, 805 AA and having an amino acid sequence having at least 90%, such as at least 95%, 96%, 97, 98%, or 99%, such as 100% sequence identity (% SI) with SEQ ID NO 1.

In one embodiment, the polypeptide comprises at least 700 amino acids, such as at least 710, such as at least 715, such as at least 718, 719, 720, 721, 722, 723 AA and having an amino acid sequence having at least 90%, such as at least 95%, 96%, 97, 98%, or 99%, such as 100% sequence identity (% SI) with SEQ ID NO 2 or SEQ ID NO 3. It may also comprise at least 715 amino acids, such as at least 716, 718, 719, 720, 721, 722, 723 AA and having an amino acid sequence having at least 99%, such as 100% sequence identity (% SI) with SEQ ID NO 2 or SEQ ID NO 3. It may also comprise at least 720 amino acids, such as at least 721, 722, 723 AA and having an amino acid sequence having at least 99%, such as 100% sequence identity (% SI) with SEQ ID NO 2. The polypeptide may also have a sequence according to SEQ ID NO 2 or SEQ ID NO 3.

In one embodiment, the peptide does not contain the signal peptide (AA 1 to 17 of SEQ ID NO 1), the transmembrane domain (AA 741 to 761 of SEQ ID NO 1) and/or the cytoplasmic domain (AA 762 to 805 of SEQ ID NO 1).

Cryo-electron microscopy structural studies suggest that the binding is between the ectodomain part of SARS-CoV-2 S protein which binds to the ACE2 N-terminal peptidase domain (PD), which is amino acids 19 to 615 of SEQ ID NO 1. Also, binding has been shown even if there are smaller sequence variations of ACE2. As such, a polypeptide comprising part of the ACE2 sequence is enough to neutralize the virus particles by binding.

Further, in table 1, SEQ ID 4 shows the extracellular subdomain I. This domain contains all reported interaction points with the spike-protein, however, the subdomain does not retain the ACE2 activity. This may be of advantage, since the peptide will not have several parallel effects, for instance making it easier to evaluate treatment results.

In one embodiment, the polypeptide comprises at least 420 amino acids, such as at least 440, such as at least 450, such as at least 458, 459,460, 461, 462, 463 AA and having an amino acid sequence having at least 90%, such as at least 95%, 96%, 97, 98%, or 99%, such as 100% sequence identity (% SI) with SEQ ID NO 4 or SEQ ID NO 5.

It has been shown that a mutation that altered the NxT/S motif in humanACE2 to a civet ACE2-like sequence (90-NLTV-93 to DAKI), expected to abolish the N-glycosylation, increased the SARS-CoV infectivity and S-protein binding (https://doi.org/10.1101/2020.04.07.024752), [16, 17]. As such, SEQ ID 3 and SEQ ID 5 represents the sequences of SEQ ID 2 and SEQ ID 4, with this mutation, respectively. As such, it is likely that these peptides will retain higher binding affinity for the spike protein.

The 2MA1 peptide (SEQ ID NO 2) will block the binding between the spike glycoprotein receptor binding domain (RBD) of the virus and the cellular receptor angiotensin-converting enzyme 2 (ACE2). In this invention, it is understood that the 2MA1 peptide functionally works within a competition assay format, making it a viable and promising virus-targeting for avoiding bio-molecule immune escape in future clinical applications.

In one embodiment, the respirable aqueous pharmaceutical composition is for use in neutralizing active virus particles binding to human Angiotensin-converting enzyme 2 (SEQ ID NO 1), such as Severe Acute Respiratory Syndrome CoronaVirus 2 (SARS-CoV-2), Severe Acute Respiratory Syndrome CoronaVirus (SARS-CoV) or Human coronavirus NL63 (HCoV-NL63) virus particles.

In one embodiment, the respirable aqueous pharmaceutical composition is for use for use in reducing the number of active virus particles being exhaled by subject infected by a virus binding to ACE2, such as SARS-CoV-2, SARS-CoV or HCoV-NL63.

Moreover, a therapeutic study in a mouse model validated that these antibodies can reduce virus titters in infected lungs. The RBD-B38 complex structure revealed that most residues on the epitope overlap with the RBD-ACE2 binding interface, explaining the blocking effect and neutralizing capacity. The results of the invention also allow to highlight the promise of the 2MA1 peptide in neutralization-based therapeutics that provides a structural basis for rational vaccine design.

In one embodiment, the respirable aqueous pharmaceutical composition is for use in the treatment or the prophylactic treatment of a patient being infected with a virus binding to human Angiotensin-converting enzyme 2 (ACE2), such as SARS-CoV-2, SARS-CoV or HCoV-NL63. The respirable aqueous pharmaceutical composition may be administered to the lung of the patient, preferably as an aerosol.

The respirable aqueous pharmaceutical composition may be successfully administered pulmonary and/or nasally to neutralize the spike glycoprotein receptor binding domain (RBD) of the virus and the cellular receptor angiotensin-converting enzyme 2 (ACE2). However, to successfully administer the respirable pharmaceutical composition, the liquid composition has got to be converted to an aerosol suitable for inhalation.

In one embodiment, the respirable aqueous pharmaceutical composition is a liquid or a liquid droplet aerosol. In one embodiment, the respirable aqueous pharmaceutical composition is has a droplet size of a median aerodynamic diameter (MMAD) of less than 10 μm, such as less than 9 μm, 8 μm, 7 μm, 6 μm or 5 μm, preferably less than 5 μm, enabling the aerosol to enter the alveoli of the lung.

In one embodiment, the respirable aqueous pharmaceutical composition is has a droplet size of a median aerodynamic diameter (MMAD) of between 0.5 to 10 μm, such as between 0.5 to 9 μm, 0.5 to 8 μm, 0.5 to 7 μm, 0.5 to 6 μm or 0.5 to 5 μm, preferably between 0.5 to 5 μm, to enter the alveoli of the lung.

The N-terminal extracellular domain of ACE2 contains 6 canonical sequons for N-linked glycosylation. Given that glycosylation can affect modulating the binding affinity, understanding the impact of glycosylation of ACE2 with respect to its binding of SARS-CoV-2 Spike glycoprotein is of high importance. As can be seen in FIGS. 8 and 9, it was found that a de-glycosylated peptide (2MA1) has worse binding characteristics than that of glycosylated 2MA1.

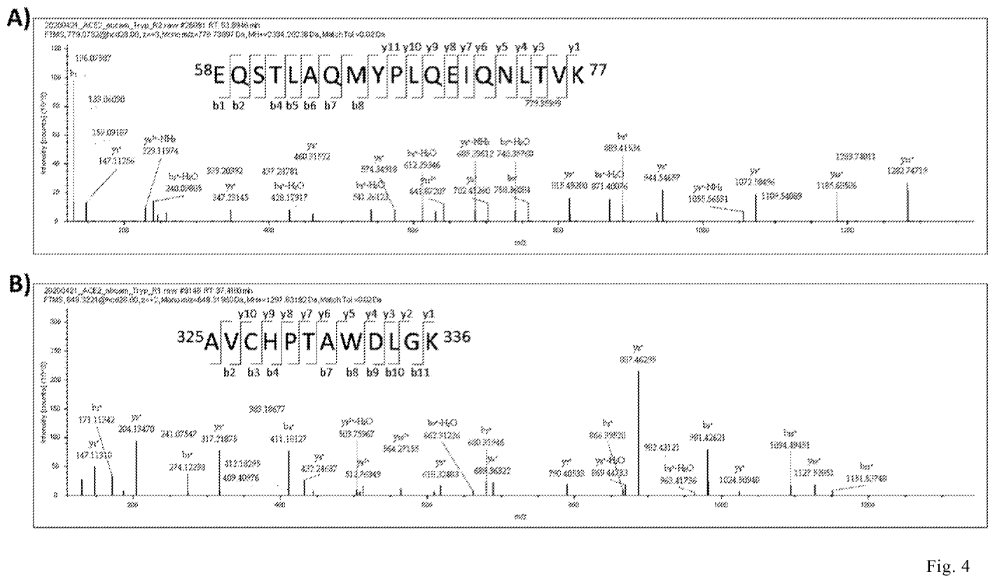

The N-terminal part together with two other regions of the 2MA1-main band protein is involved in the binding with the spike protein of the virus. One binding site was fully covered by sequencing (FIG. 4A) and the other was sequenced mostly (shown in FIG. 4B). It was verified that the protein contains the glycosylation sites, the first three glycosylation sites were sequenced by mass spectrometry as shown in FIG. 13.

It seems that these three glycosylation positions are linked to function and in the affinity interaction and complex-formation and binding of the Virus SPIKE protein. This was further confirmed using Surface Plasmon Resonance in which a microfluidic platform system was used to determine the binding kinetics with on-, and off-binding properties of the glycosylated peptide. Further, it was confirmed that these binding characteristics remain in small animal lung alveolar fluid in close conjunction to the pulmonary tissue and derived samples thereof, using sampling times of 6, 24 and 48 h, all showing the same functional binding with the active glucosylations.

Thus, in one preferred embodiment, the peptide of the invention is glycosylated. In one further embodiment, the polypeptide comprises 3 canonical N-linked glycosylation sequons. In one further embodiment, the 3 canonical N-linked glycosylation sequons are at Asparagine sites 36, 73 and 86 following the sequence numbering of SEQ ID NO 2.

2MA1 contains in total 6 canonical N-linked glycosylation sequons. The 2MA1 may be glycosylated at Asparagine sites 53, 90, 103, 322, 432, 546, and 690 (following the sequence numbering of the ACE2 full sequence (SEQ ID NO 1)).

In one embodiment, the polypeptide may comprise at least 5 canonical N-linked glycosylation sequons, such as 6 canonical N-linked glycosylation sequons. The canonical N-linked glycosylation sequons may be at Asparagine sites 53, 90, 103, 322, 432, 546, and 690 following the sequence numbering of the ACE2 full sequence (SEQ ID NO 1).

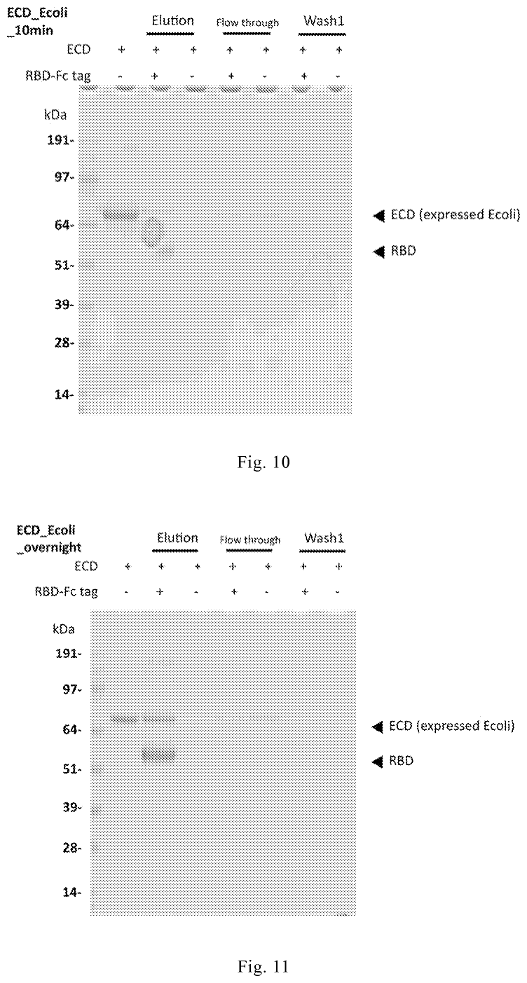

However, interestingly, as can be seen in FIGS. 10 and 11, using the formulation of the invention, the binding is improved with incubation time. As such, a de-glycosulated peptide may still be used for neutralizing virus particles binding to ACE2. De-glycolysed sequences may still have other advantages, such as simpler expression, better purity and different immune response.

Thus in one embodiment, the peptide of the invention is not glycosylated.

The peptide stability is also extremely important even after inhalation, since the stable peptides will remain be present in the nasal cavity or lungs, binding emerging virus particles, neutralizing them, thereby preventing spread to others, and keeping disease progress low to enable formation of antibodies in the host to ward off virus infection and gain immunity.

Therefore, the long term binding seen in FIG. 11 shows that virus particles in the lung will remain bound to the neutralizing peptides. This is important, since the deactivated virus particles will give the host immune system time to build up natural immunity.

Thus, in one embodiment, the composition of the invention can be used for reducing the number of active virus particles in the airways and lungs of a subject infected by a virus binding to ACE2 (such as SARS-CoV-2, SARS-CoV or HCoV-NL6).

Thereby, the inactive virus particles slow down disease progression, while enabling the subject to develop natural immunity to the virus binding to human Angiotensin-converting enzyme 2 (ACE2).

In one embodiment, lifetime of the peptide in the formulation is at least 7 days at room temperature. The lifetime at 168 h at 50 degrees can be seen in FIG. 5.

The lifetime of the peptide in the formulation makes it suitable for being loaded in a reservoir for administration. In one embodiment is provided a reservoir for a nebulizer, the reservoir comprising the respirable pharmaceutical composition. The laded reservoir may be part of a nebulizer, such as a jet nebulizer or a mesh nebulizer.

Importantly, the tests using both jet and mesh nebulizers showed that the reparable aqueous pharmaceutical composition had suitable properties for forming a stable aerosol in a reproducible and consistent manner. Also, the composition did not clog any of the nebulizers, which enabled repetitive generation of doses of aerosol with consistent properties, such as a droplet size with a median aerodynamic diameter (MMAD) suitable to enter the alveoli of the lung.

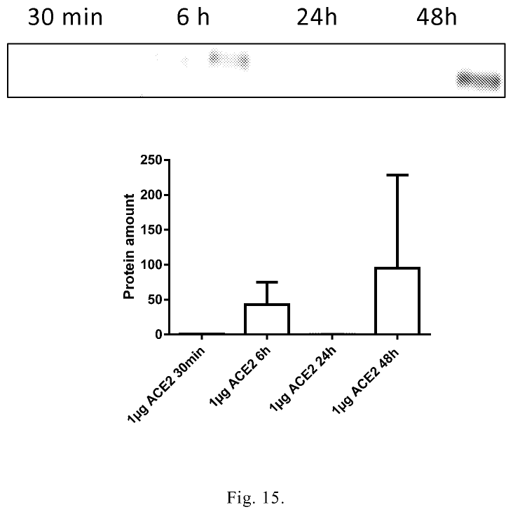

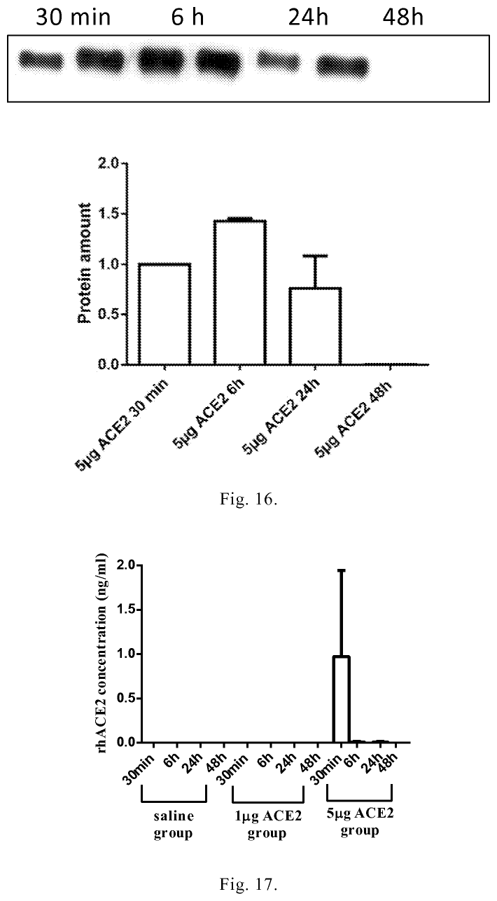

Mouse model experiments confirmed the intrapulmonary stability of ACE2 in an in vivo mouse model. As seen in FIG. 15, the peptide was barely detectable in the lungs of mice that received 1 μg ACE2 (2MA1), while it was present and very stable in the lungs of mice that were injected with 5 μg dose (FIG. 16). In both cases, the highest peptide levels were observed 6 hours after the injection. Thus, the pulmonary administered ACE2 protein analogue is stable in the lung, enabling the treatment time to take effect and neutralize the virus (virus present in the lung, newly inhaled virus, and also virus particles being generated inside the lungs, thus slowing down the virus spread).

Analysis of lung tissue using LC-MS/MS based methodology interfaced with nano-chromatography separation looked at the signal generation of both RBD (here the SARS-CoV-2 Spike S1 Receptor Binding Domain Protein) and ACE2-variant within the mouse lung after administration, with data from two consecutive lung tissue measures per time-point. It was found that the two binding partners (ACE2 peptide variant and RDBRBD) move together exactly over time, with high correlation (see FIG. 16 and table 2), they are complexed, as otherwise they would have a high degree of variation. As such, it is shown that the pulmonary administered ACE2 peptide variant interacts with the Coronavirus spike protein (RDBRBD) in vivo.

Also, plasma levels of the peptide were monitored for the mice of the mousde model experiments. ACE2 was detected only in the plasma samples of mice that received 5 μg ACE2, with decreasing concentrations overtime. The ACE2 protein was not detectable in mice that received saline or 1 μg ACE2 (FIG. 17). As such, plasma leakage is found minimal.

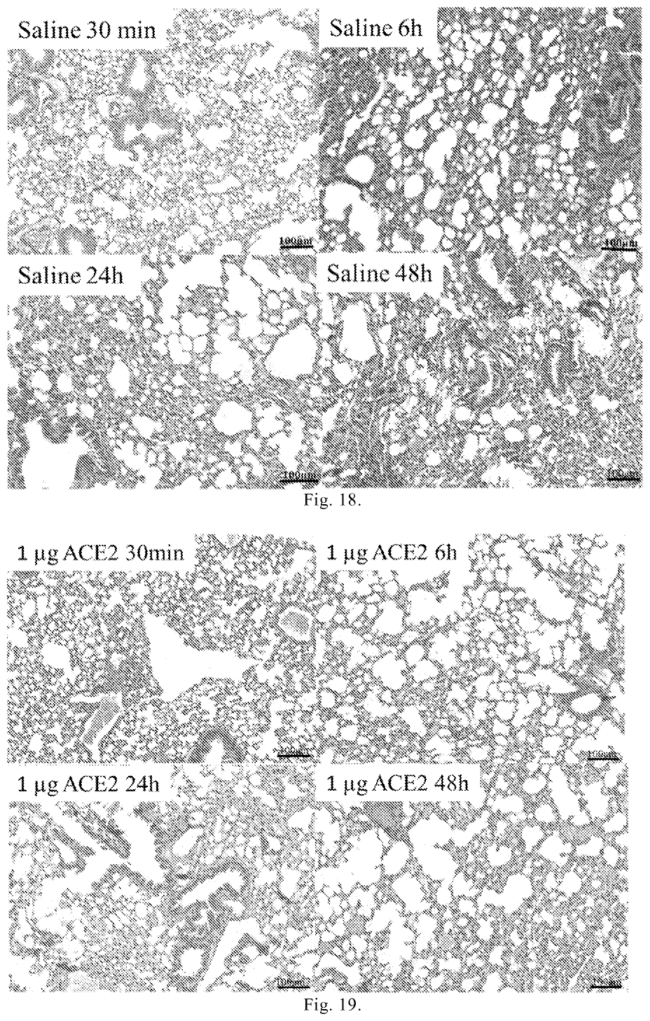

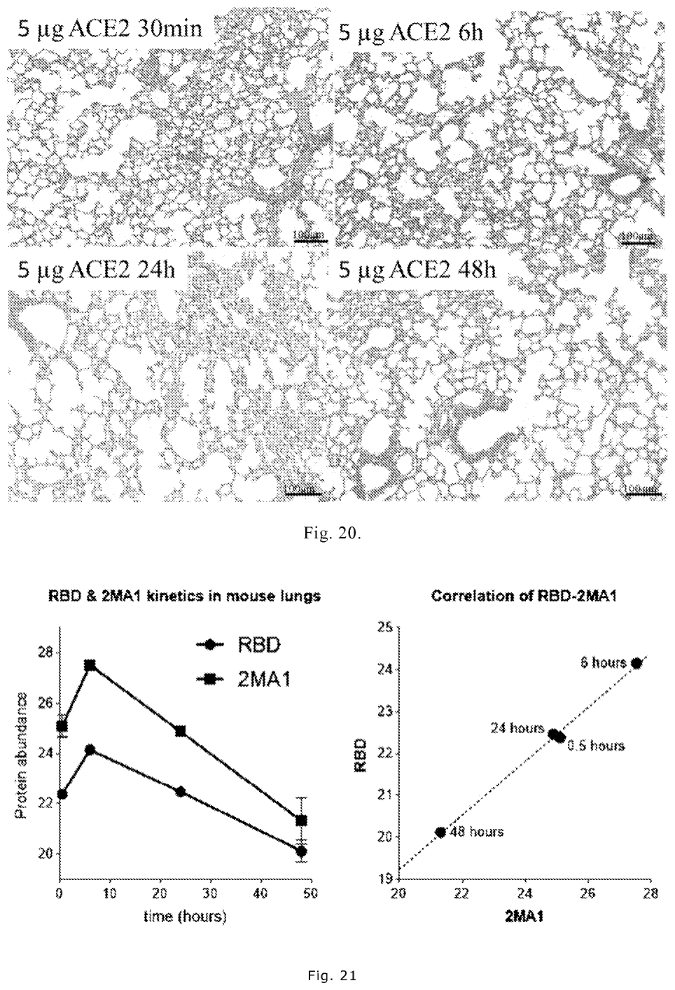

Importantly, microscopic examination of hematoxylin-eosin stainings of lungs did not show any damage or relevant difference in tissue structure between the lungs of mice that received saline, 1 μg or 5 μg ACE2 (FIGS. 18-20). This points to that the pulmonary administration of the ACE2 peptide variants does not have negative effect on pulmonary microenvironment and alveolar function the lung tissue.

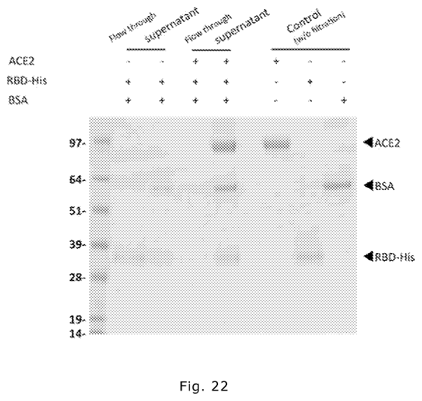

To confirm the complex formation between 2MA1 and RBD (spike protein) in the lung of the mouse, an experiment was designed to separate the complex form the non-interacting proteins. A filter of 50 kDa will discriminate between the free RBD (˜30 kDa) and the 2MA1-RBD complex (˜120 kD a). The proof of concept was performed with only RBD and the complex 2MA1-RBD pre-incubated. The FIG. 22 shows that free RBD predominantly is collected in the flow-through, while when pre-incubated with 2MA1 is only detected in the supernatant.

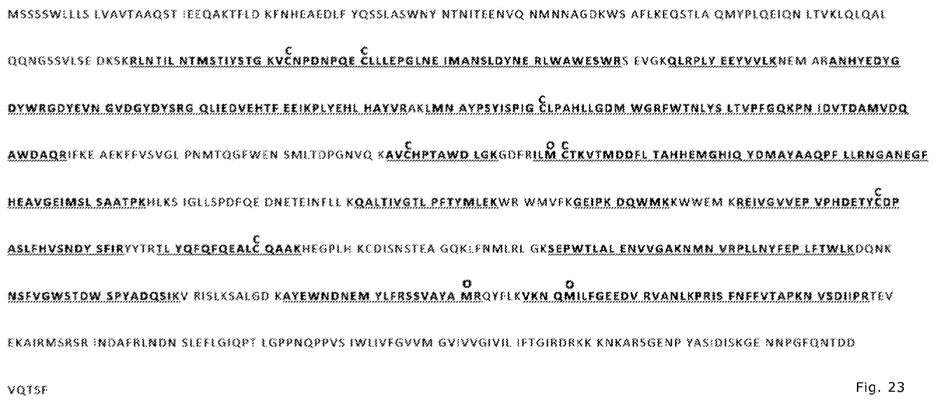

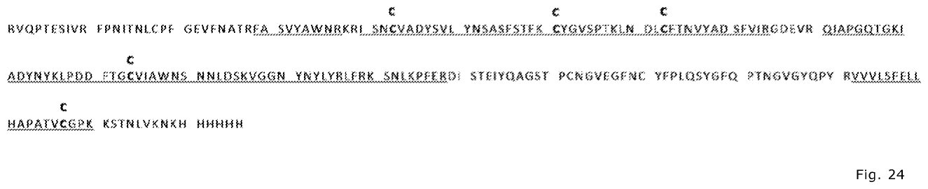

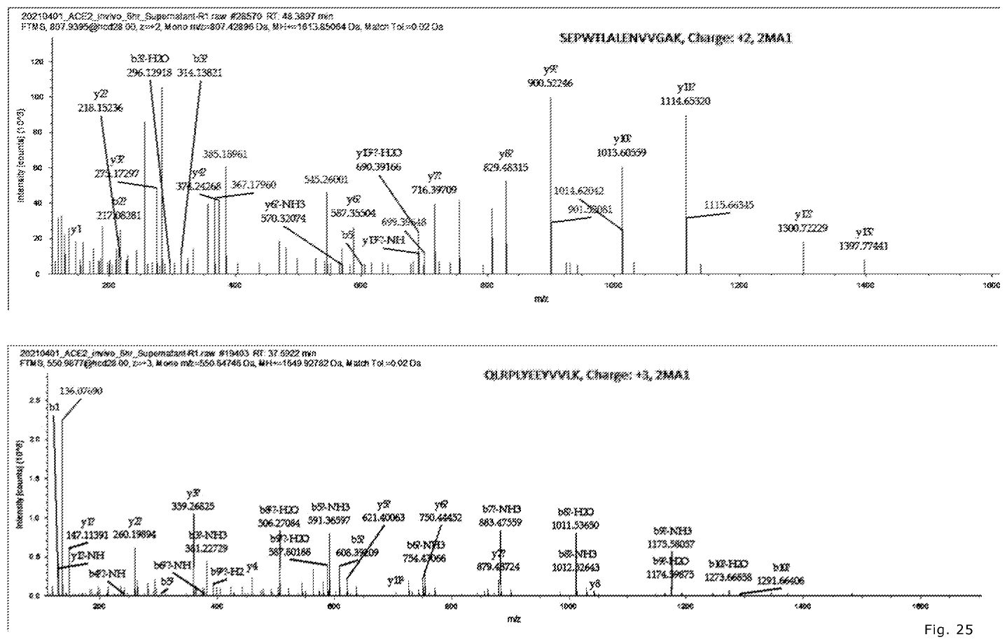

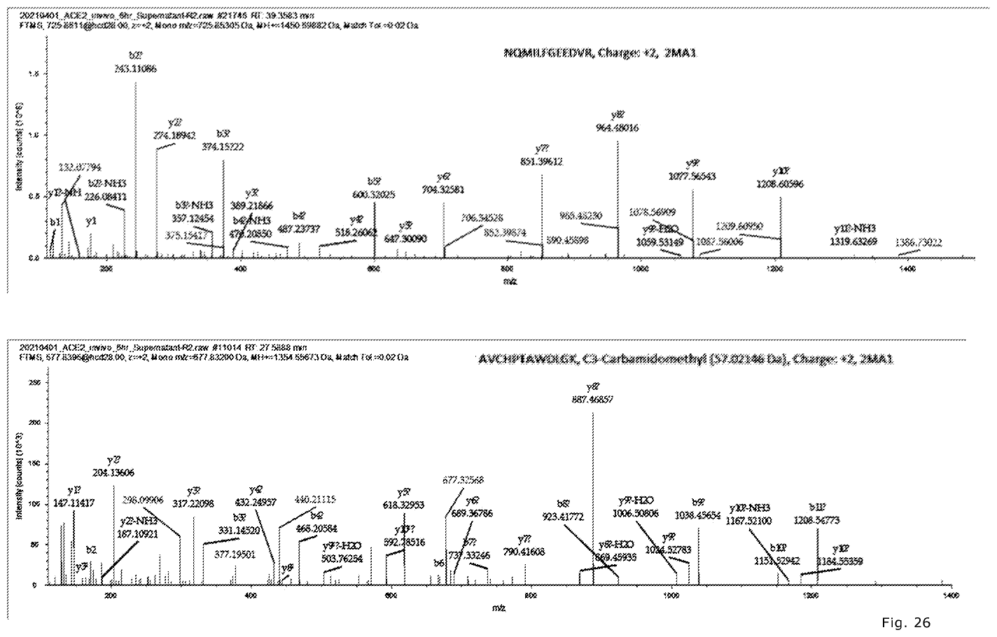

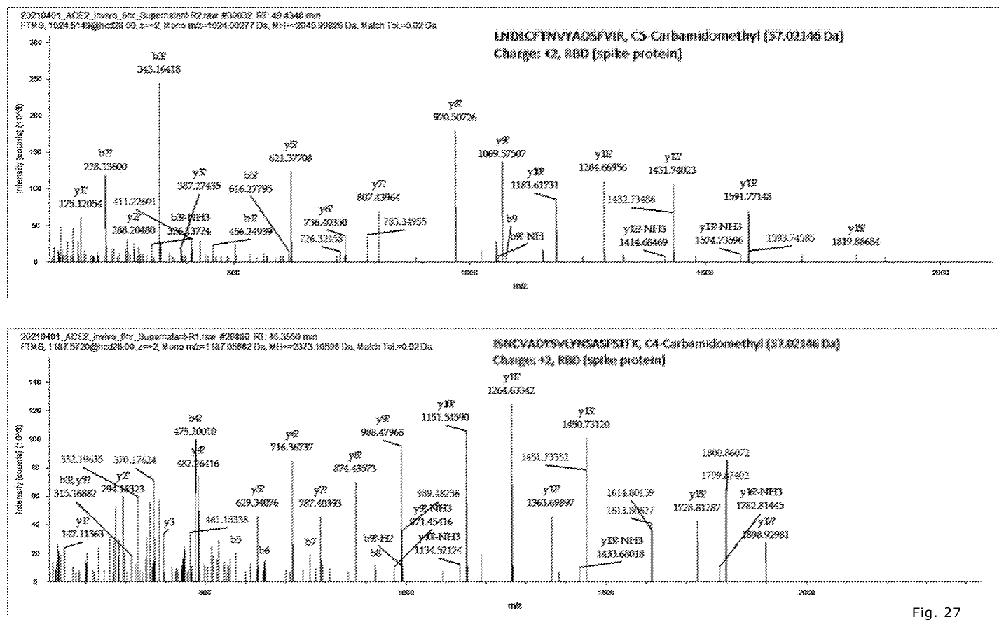

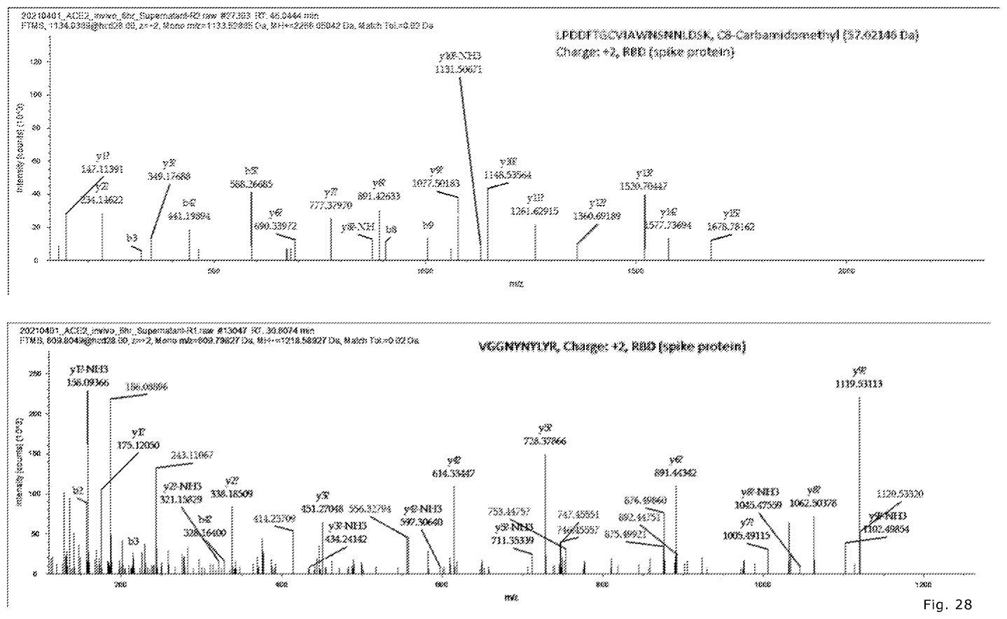

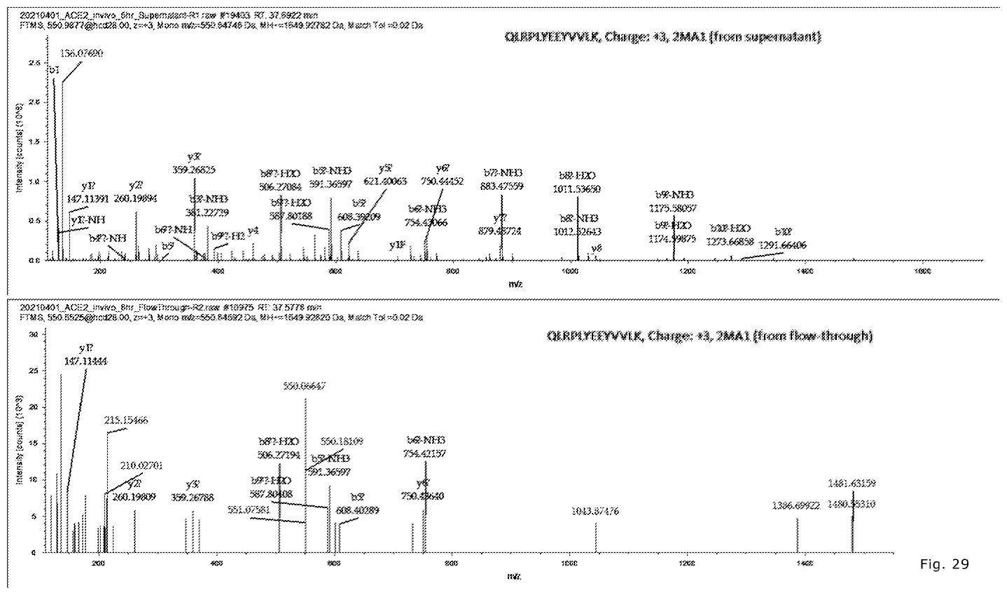

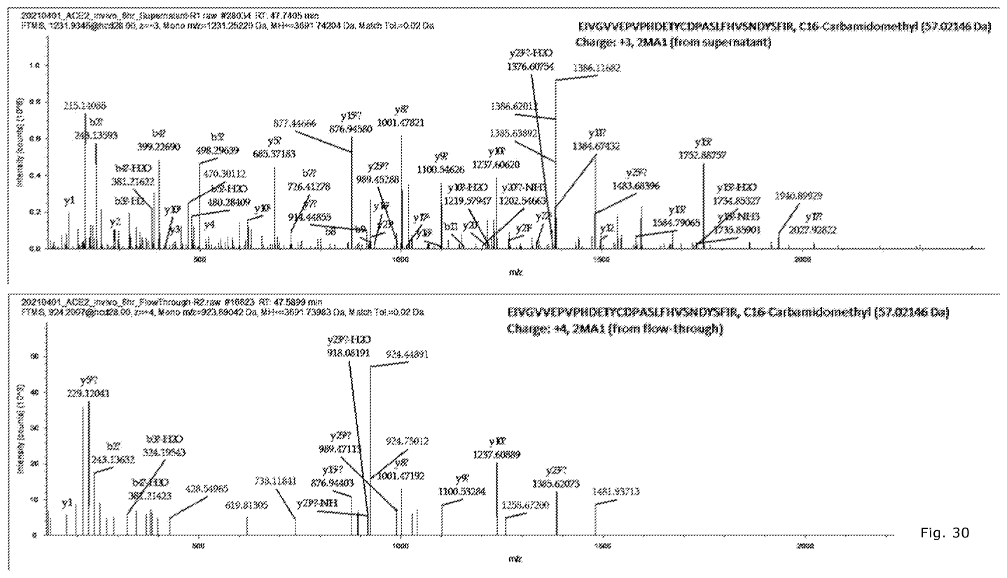

The determination of the complex formation in the lung of mice in vivo was performed at 6 hours after inoculation of mice with a mix of the two proteins (2MA1 and RBD). Proteins from lung tissue were extracted under non-denaturing conditions and submitted to the filtration process. Both the flow-through and the supernatant were immuno-precipitated for 2MA1 and RBD before processing the samples for LC-MS/MS analysis to detect the proteins. The results show that both 2MA1 and RBD were confidently identified in the supernatant. The coverage of the sequences confirmed by mass spectrometry sequencing is underlined (FIG. 23 for 2MA1 and FIG. 24 for RBD). Peptides from both extremes of the proteins were sequenced indicating in addition the integrity of both proteins after 6 hours in the lung of living mice. In total, 27 different peptides from 2MA1 and 10 from RBD were properly sequenced by mass spectrometry, covering more 55% of both of their primary structures. Four representative mass spectra showing the sequencing and the correct assignation to a peptide from the protein were selected for each of the proteins. FIGS. 25 and 26 show four mass spectra correctly assigned to peptide sequences from 2MA1. Similarly, FIGS. 27 and 28 show four spectra and their correct assignation to peptides from RBD protein. The illustrations show how the most intense signals in the spectra are explained by the sequences and assigned to y or b series of ions. The results unequivocally place the two proteins in the supernatant fraction, which strongly suggest that both proteins were in a complex in the lung of mice. The LC-MS/MS analysis of the sample collected in the flow-through did not show any detectable signal from the RBD protein, suggesting that the protein is at very low or absent in its free form in the lung. On the other hand, two weak signals were assigned to two different peptide sequences of 2MA1. The comparisons of the signal intensities and the quality of the spectra between the supernatant and in the flow-through indicate that the amount of 2MA1 detected in the flow-through is below 1% of the amount detected in the supernatant fraction (FIG. 29 and FIG. 30). The top spectra correspond to the supernatant and the bottom to the flow-through (FIGS. 29 and 30). Besides, two replicates of the experiment were performed, and the two signals from 2MA1 were only detected in one of the replicates of the flow-through.

Materials and Methods

ACE2 Extracellular Domain Expression in Ecoli

Human ACE2 (Met1-Ser740), expressed with a polyhistidine tag at the N- and C-terminus (Host E. coli) was purchased from MP biomedicals (cat #SKU 08720601). This sample is dissolved in 8 M Urea, 20 mM Tris pH8.0, 150 mM NaCl, 200 mM Imidazole, according to the manufacture's document.

Protein Characterization—after 1D Gel Separation and Gel Band Isolation

The samples were diluted in Laemmli buffer and loaded onto the 1D-gel, after the protein separation finished the proteins were stained following a Coomassie brilliant blue (CBB) protocol (alternatively silver staining can be used). A main protein band at 85 kDa consistent 2MA1 was observed and the intensity of bands correlate to amount loaded in the lane. The estimated purity was approximately 95% based on the intensity of all bands detected in the lane, made by stain-intensity determination.

Primary Sequence Confirmation

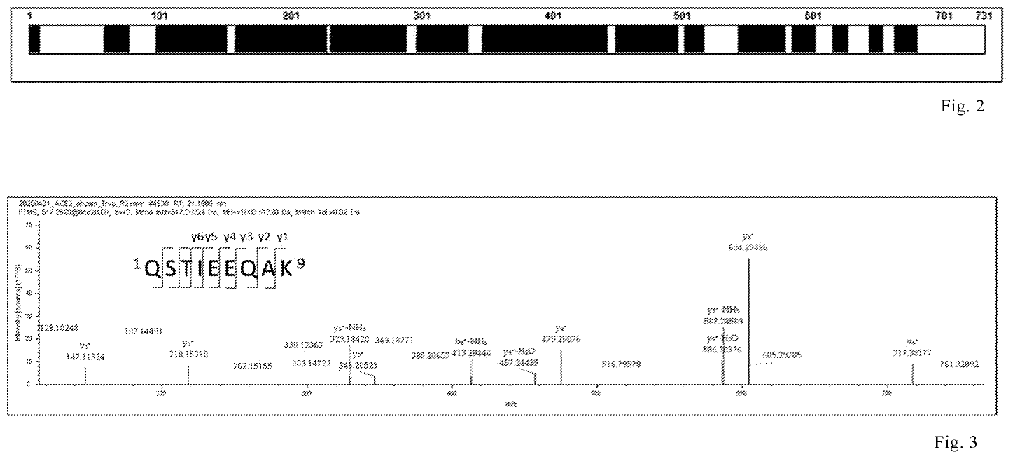

The confirmation of the 2MA1 (95% main-band) primary sequence was performed by high resolution nano-Liquid Chromatography interfaced to high resolution tandem mass spectrometry (MS/MS, a Q Exactive HF-X mass spectrometer coupled to an Ultimate 3000 RSCLnano pump (Thermo Scientific). denamed (LC-MS/MS). The samples were dissolved in ammonium bicarbonate 20 mM, trypsin was added (at a ratio of 1:10, enzyme:substrate relation), and incubated 16 hours at 37° C. The reaction was stopped by adding TFA to a final concentration of 0.1%. The mixture of peptides was next analyzed by LC-MS/MS on an Acclaim PepMap100 C18 (5 μm, 100 Å, 75 μm i.d.×2 cm, nanoViper) chromatography column stationary ohase, was used as trap column and EASY-spray RSLC C18 (2 μm, 100 Å, 75 μm i.d.×25 cm) as analytical column. Solvent A was 0.1% formic acid (FA), solvent B was 80% acetonitrile (ACN) with 0.08% FA. The flow-rate was set to 0.3 l/min and the column temperature was 45° C. The peptides were separated using a 60 min non-linear gradient and analyzed with a top 20 DDA (data dependent acquisition) method. Generated MS spectra were query to the 2MA1-Main band theoretical sequence. A total of 35 peptides covering the 71% of 2MA1 amino acid sequence were sequenced. The distribution of the 2MA sequence coverage is represented in FIG. 2.

The verification of both extremes of the protein is important for determining the integrity of the molecule. The theoretical N-terminal peptide generated by trypsin digestion is: 1QSTIEEQAK9 with a molecular mass of 1032.51 Da. From the LC-MS/MS analysis a double charged signal at 517.26 Th (1032.51 Da) was fragmented and its MS/MS was correctly assigned to the N-terminal peptide (shown in FIG. 3). The sequencing of the N-terminal peptide confirmed that the molecule preserves its N-terminal as an intact part of the molecule.

The N-terminal part together with two other regions of the 2MA1-main band protein is involved in the binding with the spike protein of the virus. One binding site was fully covered by sequencing (FIG. 4A) and the other was sequenced mostly (shown in FIG. 4B).

The protein was cloned and expressed with a His tag in the C-terminal which was used for purification. In addition, we verified that the protein contains its N-terminal intact. The LC-MS/MS analysis allowed us to confirm 71% of the sequence including totally or partially the three binding regions with the spike protein.

Stability Test of 2MA1 by SDS-PAGE Assay

The stability of 2MA1 was investigated with perspective to pH-window and sample composition.

1) SDS-PAGE Assay

As a positive control, 0.6 microgram of 2MA1 in PBS at room temperature was used.

After incubation, the reaction was stopped by adding 5 micro litres of 4× sample buffer (Thermo) and 2.22 micro litres of 0.5 M DTT.

Sample preparation; In order to prepare these samples for protein stability, applying electrophoresis assay, we performed denaturation of 2MA1, sample by heating at 95° C., for 5 min. Next, alkylation was conducted by the addition of 1.78 micro litres of iodoacetoamide (0.5 M).

2) pH-Dependence of the Stability

Sample Preparation of 2MA1
2MA1 had a concentration of 1.5 mg/mL.
2MA1 was prepared as protein solution, diluted by MilliQ water to 0.6 mg/mL.
0.6 microgram of 2MA1 were incubated in the following buffers or formulation at 50° C. for 24 hrs and 48 hrs;
1) pH 5, 100 mM citrate buffer
2) pH 7.5, PBS.
3) pH 9, 50 mM Tris-HCl
4) pH 11 buffer

3) The Stability Effect of 2MA1 in Formulation

Sample Preparation of 2MA1
2MA1 had a concentration of 1.5 mg/mL.
2MA1 was prepared as protein solution, diluted by MilliQ water to 0.6 mg/mL.
0.6 micrograms of 2MA1 were incubated in the following buffers or formulation at
50° C. for 48 hrs and 168 hrs;
1) 2MA1 in 48 h
2) 2MA1 in 168 h
3) pH 7.5, PBS, 24 h.

Contents of Formulation

Formulation was prepared with the ingredients shown in table 1, with a resulting Ph: 7.4, comprising a NaCl concentration of 8.5 mg/ml, Tween 80 concentration of 0.2 mg/ml, Phosphate buffer concentration of 0.7 mg/ml, and EDTA concentration of 0.1 mg/ml.

TABLE 1
Contents of formulation

| Materials | Concentration (mg/mL) |
|---|---|
| NaCl | 8.5 |
| Tween 80 | 0.2 |
| Phosphate Buffer | 0.7 |
| EDTA | 0.1 |

SDS-PAGE

All volume of each sample was applied to a well in the gel (NuPAGE 4-12% gel, Thermo). 3 micro litres of Seeblue2 (thermo) was used as a molecular marker. Electrophoresis ran in MOPS buffer system under 200 V for 45 min.

CBB Staining and Gel Scan

Gels were stained by using colloidal blue staining (Thermo) following the manufacturer's

instruction. In brief, after fixation of the gels, the gels were staining for 3 hr and de-stained by milliQ water over night. The gels were scanned by HP Scanjet G4050 (HP).

Surface Plasmon Resonance Spectrometry

Formulation stability and properties of 2MA1;

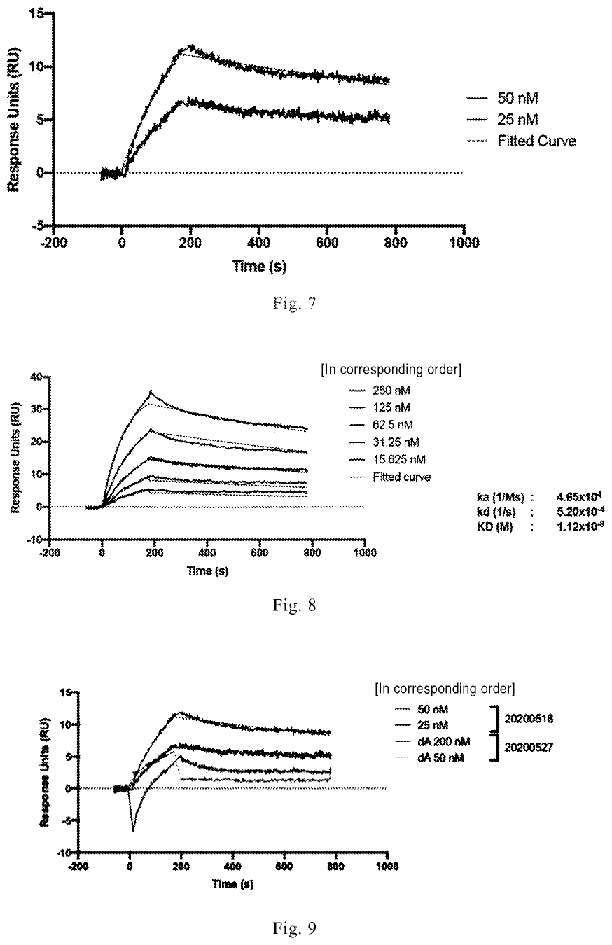

With ACE2, the full extracellular domain 18-740 amino acids, we performed the complex formation assay, identifying the binding properties of 2MA1, with the SPIKE protein of SARS COVID-2. With Surface plasmon resonance (SPR; Biacore platform), where we provide evidence of the interaction with the spike protein, at 25 and 50 nM within the graph below. The concentration dependent signal response for binding is shown in FIG. 7, which means we have the His-tag modified SPIKE protein of SARS COVID-2 bound to the SPR surface, and use the micro-fluidic platform to introduce 2MA1 to the chip surface with the immobilized SPIKE protein. The RBD-Fc was immobilized onto a CM5 micro-fluidic chip at a level of 321.4 Response units (RU). The parallel channel with in the experimental run was the blank, and acted as the reference and background, utilized for the measurements, in order to make normalizations. The 2MA1-HIS dissociate in 600 seconds.

The binding characteristics between RBD-Fc and different forms of ACE2 (ACE2-His (A-his) and deglycosilated ACE2-His (dA)) were investigated using a BIAcore X-100 instrument (GE Healthcare, Uppsala, Sweden). RBD-Fc was immobilized on a CM5 sensor chip (GE Healthcare) at a level of 321.4 response units (RU) using standard amine coupling. In parallel, one flow cell was incubated with buffer alone (i.e. without RBD-Fc), serving as control.

Interaction experiments were performed with injections of 15.625, 31.25, 62.5, 125, 250 nM of ACE2-His and deglycosilated ACE2-His in running buffer (0.02 M phosphate buffer with 2.7 mM KCl, 0.137 M NaCl and 0.05% Solubilizer P20 (Tween 20)) at a flow rate of 30 l/min for 180 seconds. After the end of each injection, dissociation was performed for 600 seconds and then the surface is regenerated with 10 mM Glycine HCl pH 2.5 for 30 seconds, followed by a 10 seconds washing procedure. After X and Y normalization of data, the blank curves from the control flow cell of each injected concentration were subtracted. The BIA evaluation 3.1 analysis software (GE Healthcare) was used to determine equilibrium dissociation constants (KD) from the processed data sets by fitting to a 1:1 molecular binding model. The binding data can be seen in FIG. 8.

2MA1d—de-glycosylated binding is seen in FIG. 9. In conclusion, 2MA1d (the deglycosylated form of the protein) is weak binding with RBD compared to 2MA1. We run the binding assay for deglycosilated ACE2-His (dA) using 50 and 200 nM of dA as compared to the 25 and 50 nM of ACE2-His (A-His) used on 18th. From this sensogram overlay it was deduced that the binding of dA to RBD-Fc is drastically reduced than the native A-His form.

Intrapulmonary Injection of ACE2 and RBD of the Viral Spike Protein

Soluble rhACE2 (Abcam, Cat. No: ab151852) was dissolved in saline and injected in the lungs of BDF1 mice at two doses (1 and 5 μg protein in 200 μl saline) via tracheostoma. Control animals received only solvent.

The RBD of the viral spike protein (Acro Biosystems, Cat. No: SPD-C52H3) and ACE2 were dissolved, mixed in 1:1 molar ratio and injected immediately into the lungs of mice as described above.

Preparation of Tissue Samples

Lungs were harvested 30 min, 6, 24 and 48 hours after the injection and lung lobes were either frozen in liquid nitrogen for Western blot analysis or fixed in formalin and embedded in paraffin for histological analysis.

For collecting plasma from the inner corner of the eye, haematocrit capillaries with sodium-heparin (Deltalab, cat.no. 7401) and 1 ml MiniCollect K3E K3EDTA tubes (Greiner-BioOne, cat.no. 450474) were used. Blood samples of mice (˜200-400 μl) were centrifuged at 1500 rpm for 10 minutes at 4° C., the supernatant was piped into Eppendorf-tubes (˜200 μl), frozen in liquid nitrogen and stored at −80° C. for further investigation.

Western Blot Analysis of Intrapulmonary ACE2 Levels

The lobes of the lungs were homogenized manually with a glass homogenizer in 400 μl Pierce RIPA buffer (Thermo Fisher Scientific, cat.no. 89900) per sample supplemented with 4 μl Protease Inhibitor Cocktail (Sigma-Aldrich, cat.no. P8340), 4 μl 0.5M EDTA (Thermo Fisher Scientific, cat.no. 15694), 8 μl 100 mM-os phenylmethanesulfonyl fluoride in absolute ethanol (Sigma-Aldrich, cat.no. P7626) right before use. Pierce BCA Protein Assay Kit (Thermo Fisher Scientific, cat.no. 23225) was used for determination the protein concentration of the samples in the accordance with the manufacturer's manual. 10 μg protein per sample was loaded for the SDS-polyacrylamide gel electrophoresis. The proteins were electro-transferred onto nitrocellulose membranes. After blotting, the membranes were blocked in 5% BSA/TBS-Tween for one hour. then incubated them with human ACE2 antibody (Invitrogen. cat. no. PA5-110613) in dilution 1:2000 overnight. Blots were then incubated with anti-rabbit (H+L) secondary antibody (Thermo Fisher Scientific. cat.no. 31460) and signals were detected by using SuperSignal WestFemto Maximum Sensitivity Substrate (Thermo Fisher Scientific. cat.no. 34095).

Investigation of Plasma ACE2 Levels by Enzyme-Linked Immunosorbent Assay (ELISA)

Human ACE2 ELISA kit was purchased from RayBiotech (cat.no. ELH-ACE2). All plasma samples and kit components were equilibrated to room temperature before the measurement. Sample preparation and detection procedures were performed in the accordance with the manufacturer's manual. The detection range of the assay is 0.025 ng/ml-20 ng/ml. The absorbance was determined at 450 nm with Multiskan Sky microplate reader (Thermo Fisher Scientific. cat.no. 51119600).

Protein Extraction and Enrichment from Tissue Samples

Mouse lung tissue samples were ground on ice was until the tissues were broken. 300 μL of lysis buffer (100 mM Sodium-Phosphate, pH 8.0, 600 mM NaCl, 0.02% Tween-20) was added to the broken tissue, followed by sonication by bioruptor (15 sec ON, 15 sec OFF, 40 cycles. This process ran twice). The sonicated sample was centrifugedg at 20,000×g for 3 min @ 4° C.

After centrifugation, supernatant was moved to new 1.5 mL tube. 280 uL of sample was taken out and mixed with 280 μuL of milliQq water (to make 1× binding buffer—Use his-tag dynabeads 50 μuL (follow the manufacture's instruction), followed by incubation with rotation 5 min in the cold room. Thereafter, it was kept on a magnet for 2 min and supernatant was discarded, followed by 4 washes with 1× binding buffer (300 uL). At the 5th wash, 50 mM phosphate buffer (200 μuL) was used and the supernatant was removed.

Digestion

50 uL of 100 mM Ambic was added to the Dynabeads which bind with protein samples, from the protein extraction and enrichment step (above).

1 ug of trypsin (stock solution 1 mg/mL was added and samples were digested for 18.5 hrs at 37° C. @ 500 rpm.

Next day—1.2 uL of 5% TFA (final concentration should be 0.1%) to was added to each sample.

Samples were treated at 95° C. for 5 min @500 rpm, after which samples were spun down. magnet for 2 min to separate the samples from dynabeads and transfer supernatant was transferred to new 1.5 mL tube. This step was repeated to get all sample. Supernatant was speed vac for 1 hr (40-50 min). 20 uL of 0.1% TFA was added to the dried sample to resuspend it, followed by centrifuge at 20,000×g for 3 min. The resulting supernatant was moved to new MS vial for analysis.

Analysis

The identification and quantification of ACE2 (here 2MAI) was is performed by mass spectrometry that is based on a nano-separation chromatography liquid phase separation platform. The separation is interfaced with high-resolution mass spectrometry, utilizing Orbitrap technology. The assay will provides quantitative high-resolution, accurate-mass (HRAM) liquid chromatography mass spectrometry (LC-MS) with record-setting performance with the power of built-in software features, which provide elevated sensitivity and selectivity. The Orbitrap technology also delivers depth of analysis to trace levels (attomole level) with high quantitative accuracy and precision.

Sample Preparation; The protein product was dissolved in ammonium bicarbonate 50 mM and digested with trypsin at a 1:10 m:m ratio (enzyme:protein). The enzyme was added and the reaction was incubated for 16 h at 37° C. The reaction was stopped by adding TFA to a final concentration of 0.5%.

Next processing step 1; the generated peptides were analyzed in duplicates by LC-MS. The method of choice MS analysis is usually, but not necessarily Data Dependent Acquisition (DDA) on high-resolution mass spectrometer (HF-X, Thermo). Usually from the MS1 scan the top 20 signal are selected for MS2 fragmentation and excluded for 40 s to be selected again. The normalized collision energy (NCE) is usually fix to 28%. The chromatographic conditions for the separation of peptides usually involve a 1 h non-linear elution gradient for the recommended trap and analytical columns, Acclaim PepMap100 C18 (5 μm, 100 Å, 75 μm i.d.×2 cm, nanoViper) and EASY-spray RSLC C18 (2 μm, 100 Å, 75 μm i.d.×25 cm) respectively.

Next processing step 2; the acquired raw files were submitted to peptide and protein identification. The raw files were are processed, but not necessarily with the Proteome Discoverer software (Thermo). The peptides and proteins in the samples were identified by matching the spectra with a human protein database, usually but not necessarily downloaded from UniProt repository. The search engine of choice is was usually the Sequest, which is was provided together with the Proteome Discoverer. The peptides and proteins identified in the samples are were reported using a cutoff for positive identification controlling the FDR at 1%.

Data analysis; Search protein sequence by Proteome Discoverer using fasta file (all mouse sequence and human ACE2, and S-protein sequence)

For the quantification of ACE2 and RBD, the value from “protein abundances” that is simple summation of its associated and used peptide group abundances was used. The value was normalized by log 2 and compared between the samples.

For detection and identification of resulting outcomes, CBB staining and Colloidal Blue Stain kit (Thermo) was used following manufacture's instructions.

Mouse Lung Protein Complex Confirmation Experiments

In order to confirm the complex formation; “ACE2-S Protein”, a sample preparation step was introduced. The ultra-filtration procedure with a 50 k Da cut-off (AmiconUltra-0.5 device), was introduced for the recombinant RBD, with and without the ACE2 protein.

To form the “2ACE2-RBD” complex, an incubation was performed of these two proteins for 10 min at room temperature.

In order to prepare the filtration procedure, a preparation was performed prepared, by pre-activation preparation of the filtration device as follows;
Prewash and blocking by BSA 0.1 mg/ml) by the use of 500 μuL of milli Q water.
Centrifuge at 14,000×g for 10 min. Discard flow through fraction.
After pre-wash, to block the non-specific binding of proteins, add around 500 μL of BSA (50 μg in MilliQ) sample to the Amicon Ultra filter device and cap it. Place capped filter device into the centrifuge rotor, aligning the caps trap toward the center of the rotor; counter balance with a similar device. Spin the device at 14,000×g for approximately 10 min.

After these steps, the flow through fraction was discarded, where the RBD-His is eliminated (See FIG. 21).

After pre-wash, to block the non-specific binding of proteins, around 500 μL of BSA (50 ug in MilliQ) sample was added to the Amicon Ultra filter device and it was capped. The capped filter device was placed into the centrifuge rotor, aligning the caps trap toward the center of the rotor; counter balance with a similar device. The device was spun at 14,000×g for approximately 10 min.

The flow through fraction was discarded. The filter device was rinsed by pipetting with 500 μL of MilliQ water, which was discarded.

Next, load of sample (RBD or ACE2-RBD complex) to the filtration device was made, followed by centrifugation of the device. After centrifugation, speed vac was used to dry out the samples. Then, add 1×SDS sample buffer and reduce the proteins, and run SDS-PAGE gel separation and stain the gel by CBB (shown in FIG. 21).

The filter Sample preparation procedure worked well in isolating the free RBD-His protein, not being complexed by ACE2. The free RBD go into the flow-through fraction. On the other hand, free RBD didn't go into it. The protein complex stayed in the filtration device.

Sample Processing of RBD-his—ACE2 Protein Complex from Mouse Lung Tissue by Liquid Chromatography-Mass Spectrometry Analysis

To confirm the presence of ACE2-S protein complex within mouse lung tissue, 50 k Da cut filtration (AmiconUltra-0.5 device) was used for the lung tissues extracts treated with ACE2-S protein. Two piece of mouse lungs (6 hr treatment) were used. The size of each piece was almost a quarter of the left lung.

Protein extraction was used with 300 uL of lysis buffer (100 mM Sodium-Phosphate, pH 8.0, 600 mM NaCl, 0.02% Tween-20) and Sonicated by bioruptor (15 sec ON, 15 sec OFF, 40 cycles. This process ran twice). After centrifugation at 20,000×g for 3 min @ 4° C.

For the preparation of the filtration device, a prewash was conducted, using 500 μL of milli Q water. Centrifuge at 14,000×g for 10 min. Discard flow through fraction.

After pre-wash, to block the non-specific binding of proteins, around 500 μL of BSA (50 μg in MilliQ) sample was added to the Amicon Ultra filter device and it was capped. The capped filter device was placed into the centrifuge rotor, aligning the caps trap toward the center of the rotor; counter balance with a similar device. Spin the device at 14,000×g for approximately 10 min.

The flow through fraction was discarded, followed by a rinse of the filter device by pipetting with 500 μL of MilliQ water, which was discarded.

The supernatant from the protein extracts was taken to the prepared filtration devices. The device was spun at 14,000×g for approximately 10 min. The flow through fraction was collected to new 1.5 mL tube. 100 μL Milliq water was added to the filter divicedevice. To recover the concentrated solute, the Amicon®Ultrafilter device was placed upside down in a clean microcentrifuge tube. It was spun for 2 minutes at 1,000×g to transfer the concentrated sample from the device to the tube. The ultrafiltrate couldan be stored in the centrifuge tube. Transfer eEach sample fraction was transferred to 1.5 mL tube, adding equal volume of milliQq water (to adjust the concentration to 1× binding buffer)

For Pull down assay, use his-tag dynabeads were used (5 μL) for each flow through and supernatant fraction. Incubation was performed with rotation for 5 min in the cold room. Keep it on tThe magnet was kept for 2 min and then discard the supernatant was discarded. Wash 4 times washing was performed next with 300 μL of 1× binding buffer (50 mM Phosphate buffer pH 8.0, 300 mM NaCl, 0.01% Tween-20). At the 5th wash, use 100 mM Ambic (200 μL) was used and Transfer the beads-protein complex was transferred to new tube. Remove Supernatants were removed.

Add 50 μL of 100 mM Ambic to the Dyna-beads which bind with protein samples

For reduction the below protocol was followed:
Add 1 uL of 250 mM DTT to sample, and heat 95° C. for 5 min (final conc.: around 5 mM) Use magnet and take supernatant.

To alkylate the proteins, add 1 uL of 250 mM IAA to sample, and for 30 min in dark (final conc.: around 5 mM).

For digestion, add 1 ug of trypsin (stock solution 1 mg/mL)+10 uL of 100 mM Ambic. For digestion during 16 hrs at 37° C. @ 500 rpm:
Add 1.2 μL of 5% TFA (final concentration should be 0.1%) to each sample
Spin down the sample and Speed vac for 1 hr (40-50 min)
Add 20 μL of 0.1% TFA to dried sample and resuspend it
Centrifuge at 20,000×g for 3 min
Take supernatant to new MS vial
Run MS and analyze data by proteome discoverer

MS Data Analysis

MS data analysis showed that 2MA1 and RBD were abundantly identified in the supernatant fraction. The sequence coverage of both proteins were over 55%.

In addition, within the flow through fraction, we were not able to identify the reliable signals of the 2MA1.

The signal response in the flow through fraction was low as compared to the supernatant.

The sequence fragments found are shown in FIGS. 23 and 24, MS spectra for the 2MA1 fragments are shown in FIGS. 25 and 26, and MS spectra for RBD fragments are shown in FIGS. 27 and 28. Comparison spectra between supernatant and filtration are shown in FIGS. 29 and 30.

Results

Stability Test of 2MA1 by SDS-PAGE Assay

The Effect on Different pH on 2MA1

Stability of ACE2 was investigated by different buffer conditions between pH 5 and 11. FIG. 5 demonstrated the pH read-out effects of the stability test. Except for the positive control, gel staining showed extra protein bands upper side of the gel (above 190 kDa). This result indicates that heated ACE2 may have aggregation and/or dimerization. On the other hand, protein form of ACE2 such as degradations were not observed at the lower molecular weight.

In addition, signal intensity of ACE2 at pH 11 was lower than that of others with only approx., 6% after 168 h. According to the European pharmacopoeia 10.0, the pH of liquid preparations of nebulization is not lower than 3 and not higher than 10.

The 2MA1 peptide seems to be stable within the pH window of pH 5-9.5.

2MA1—Main Product Characterization

It was verified that the protein contains its N-terminal intact. The LC-MS/MS analysis allowed us to confirm 71% of the sequence including totally or partially the three binding regions with the spike protein.

A total of 35 peptides covering the 71% of 2MA1 amino acid sequence were sequenced. The distribution of the 2MA sequence coverage is represented in FIG. 2.

The theoretical N-terminal peptide generated by trypsin digestion is: 1QSTIEEQAK9 with a molecular mass of 1032.51 Da. From the LC-MS/MS analysis a double charged signal at 517.26 Th (1032.51 Da) was fragmented and its MS/MS was correctly assigned to the N-terminal peptide (shown in FIG. 3). The sequencing of the N-terminal peptide confirmed that the molecule preserves its N-terminal as an intact part of the molecule.

The N-terminal part together with two other regions of the 2MA1-main band protein is involved in the binding with the spike protein of the virus. One binding site was fully covered by sequencing (FIG. 4A) and the other was sequenced mostly (shown in FIG. 4B).

It was verified that the protein contains the glycosylation sites described above, as the glycosylated form. The first three glycosylation sites were sequenced by mass spectrometry as shown in FIG. 13.

Binding of ACE2 Extracellular Domain Expressed in Ecoli at 10 Min and 10 h Incubation

In both 10 min and 10 h incubation, samples (FIGS. 11 and 12), the protein enabled to interact with RBD. In the flow through fraction, the protein in 10 min reaction was detected in flow through fraction. In contrast, protein in overnight reaction sample, wasn't. This result shows that not all of the protein interacted with RBD for 10 min. To overcome numbers of the interacted proteins (ACE2-RBD complex), it is necessary to increase the reaction time.

Intrapulmonary Stability of ACE2 in an In Vivo Mouse Model

The protein was barely detectable in the lungs of mice that received 1 μg ACE2 (FIG. 15), while it was present and very stable in the lungs of mice that were injected with 5 μg dose (FIG. 16). In both cases, the highest protein levels were observed 6 hours after the injection.

Analysis of Circulating ACE2 (2MAI) Levels

We detected ACE2 only in the plasma samples of mice that received 5 μg ACE2, with decreasing concentrations over time. The ACE2 protein was not detectable in mice that received saline or 1 μg ACE2 (FIG. 17).

Histological Evaluation of Lung Tissue Samples

Microscopic examination of hematoxylin-eosin stainings of lungs did not show any damage or relevant difference in tissue structure between the lungs of mice that received saline, 1 μg or 5 μg ACE2 (FIG. 18-20).

Kinetic Profiling of RBD and ACE2-Variant in mMouse Tissue

What we can prove here is that by quantifying the signal generation of both RBD and ACE2-variant within the mouse 1Lung after administration, with data from two consecute lung tissue analyses measures per time-point.

TABLE 2
Kinetic profiling of RBD and ACE2-variant

| Pearson r | |
|---|---|
| R | 0.9980 |
| 95% confidence interval | 0.9038 to 1.000 |
| R squared | 0.9960 |
| P value | |
| P (two-tailed) | 0.0020 |
| P value summary | ** |
| Significant? (alpha = 0.05) | Yes |
| Number of XY pairs | 4 |

Since the two binding partners move together exactly over time, with high correlation (see FIG. 21 and table 2 above for correlation), they are complexed, as otherwise they would have a high degree of variation.

The assay is a LC-MS/MS based methodology interfaced with nano-chromatography separation.

Mouse Lung Protein Complex Confirmation Experiments

In FIG. 22, the result from the sample preparation step using a 50 k Da cut filtration (AmiconUltra-0.5 device) are shown. Isolated free RBD-His protein, not being complexed by ACE2, go into the flow-through fraction (first two columns). On the other hand, the protein complex stayed in the filtration device (second two columns). As such, the 50 k Da cut filtration method could be used to probe complex formation in lung tissue.

Sample Processing of RBD-His—ACE2 Protein Complex from Mouse Lung Tissue by Liquid Chromatography-Mass Spectrometry Analysis

To confirm the presence of ACE2-S protein complex within mouse lung tissue, the 50 k Da cut filtration was used for the lung tissues extracts treated with ACE2-S protein. The 2 pieces of mouse lungs (from the 6 hr treatment experiments) were used.

MS analysis of the revealed that 2MA1 and RBD were abundantly identified in the supernatant fraction. The sequence coverage of both proteins were over 55%. In total, 27 different peptides from 2MA1 and 10 from RBD were properly sequenced by mass spectrometry. Four representative mass spectra showing the sequencing and the correct assignation to a peptide from the protein were selected for each of the proteins. FIGS. 25 and 26 show four mass spectra correctly assigned to peptide sequences from 2MA1. Similarly, FIGS. 27 and 28 show four spectra and their correct assignation to peptides from RBD protein. The illustrations show how the most intense signals in the spectra are explained by the sequences and assigned to y or b series of ions. The results unequivocally place the two proteins in the supernatant fraction, which strongly suggest that both proteins were in a complex in the lung of mice. In addition, within the flow through fraction, we were not able to identify the reliable signals of the 2MA1. The signal response in the flow through fraction was low as compared to the supernatant. The identified protein fragment sequences are shown in FIGS. 23 and 24. MS spectra for the 2MA1 fragments are shown in FIGS. 25 and 26, and MS spectra for RBD fragments are shown in FIGS. 27 and 28. Comparison spectra between supernatant and filtration are shown in FIGS. 29 and 30. The top spectra correspond to the supernatant and the bottom to the flow-through (FIGS. 29 and 30).

As such, it is clear that the 2MA1 (ACE2) is in complex in the lung tissue samples. Furthermore, the sequence analysis confirms that the complexes are ACE2-RBDDS complex. Finally, the sample preparation step indicates that the complexes were stable for 6 hours in the lung tissue.

1. A respirable aqueous pharmaceutical composition, comprising a neutralizing affinity binder for a virus binding to angiotensin-converting enzyme 2 (ACE2), a buffer, and a solubilizer.

2. The respirable aqueous pharmaceutical composition according to claim 1, wherein the composition is a liquid or a liquid droplet aerosol; preferably the composition being a liquid droplet aerosol.

3. The respirable aqueous pharmaceutical composition according to any one of claims 1 to 2, having a pH of 5 to 9.5, preferably 6 to 8.

4. The respirable aqueous pharmaceutical composition according to any one of claims 1 to 3, wherein the buffer is selected from the group consisting of citrate buffer, phosphate buffer, phosphate-buffered saline (PBS), MOPS (3-(N-morpholino)propane sulfonic)acid) buffer, HEPES (4-(2-hydroxyethyl)-1-piperazineethanesulfonic acid) buffer, DIPSO (3-(N,N-Bis[2-hydroxyethyl]amino)-2-hydroxypropanesulfonic acid) buffer, Tris-HCl buffer or a combination of these.

5. The respirable aqueous pharmaceutical composition according to any one of claims 1 to 4, wherein the buffer concentration is at least 10 mM, such as from 10 to 250 mM, such as from 50 to 200 mM, such as 50 to 100 mM.

6. The respirable aqueous pharmaceutical composition according to any one of claims 1 to 5, wherein the solubilizer is a non-ionic solubilizer.

7. The respirable aqueous pharmaceutical composition according to any one of claims 1 to 6, wherein the solubilizer is a non-ionic polymeric solubilizer.

8. The respirable aqueous pharmaceutical composition according to any one of claims 1 to 7, wherein the solubilizer is selected from the group consisting of Polysorbate 20 (polyoxyethylene (20) sorbitan monolaurate), Polysorbate 40 (polyoxyethylene (20) sorbitan monopalmitate), Polysorbate 60 (polyoxyethylene (20) sorbitan monostearate), Polysorbate 80 (Polyoxyethylene (20) sorbitan monooleate), a poloxamine solubilizer or a polysaccharide solubilizer or a combination of these.

9. The respirable aqueous pharmaceutical composition according to any one of claims 1 to 8, wherein the solubilizer concentration is 0.01 to 0.8 mg/ml, such as 0.05 to 0.5 mg/ml, such as 0.1 to 0.4 ml/ml, such as 0.2 mg/ml.

10. The respirable aqueous pharmaceutical composition according to any one of claims 1 to 9, further comprising a chelating agent.

11. The respirable aqueous pharmaceutical composition according to claim 10, wherein the chelating agent is selected from the group consisting of ethylene diaminetetraacetic acid (EDTA), nitriloacetic acid (NTA), phospohates, citric acid, and glycine.

12. The respirable aqueous pharmaceutical composition according to claim 10 or 11, wherein the concentration of the chelating agent in the composition is 0.01 to 0.8 mg/ml, such as 0.05 to 0.5 mg/ml, such as 0.1 to 0.2 ml/ml, such as 0.1 mg/ml.

13. The respirable aqueous pharmaceutical composition according to any one of claims 1 to 12, wherein the neutralizing affinity binder is a polypeptide comprising at least 770 amino acids, such as at least 780, such as at least 790, such as at least 800, 801, 802, 803, 804, 805 AA and having an amino acid sequence having at least 90%, such as at least 95%, 96%, 97, 98%, or 99%, such as 100% sequence identity (% SI) with SEQ ID NO 1.

14. The respirable aqueous pharmaceutical composition according to any one of claims 1 to 12, wherein the neutralizing affinity binder is a polypeptide comprising at least 700 amino acids, such as at least 710, such as at least 715, such as at least 718, 719, 720, 721, 722, 723 AA and having an amino acid sequence having at least 90%, such as at least 95%, 96%, 97, 98%, or 99%, such as 100% sequence identity (% SI) with SEQ ID NO 2 or SEQ ID NO 3.

15. The respirable aqueous pharmaceutical composition according to claim 13 or 14, wherein the polypeptide comprises 3 canonical N-linked glycosylation sequons.

16. The respirable aqueous pharmaceutical composition according to claim 15, wherein the 3 canonical N-linked glycosylation sequons are at Asparagine sites 36, 73 and 86 following the sequence numbering of SEQ ID NO 2.

17. The respirable aqueous pharmaceutical composition according to claims 13 to 16, wherein the polypeptide comprises at least 5 canonical N-linked glycosylation sequons, such as 6 canonical N-linked glycosylation sequons.

18. The respirable aqueous pharmaceutical composition according to claim 17, wherein the canonical N-linked glycosylation sequons are at Asparagine sites 53, 90, 103, 322, 432, 546, and 690 following the sequence numbering of the ACE2 full sequence (SEQ ID NO 1).

19. The respirable aqueous pharmaceutical composition according to any one of claims 13 to 18, wherein the polypeptide concentration is at least 0.1 nM, such as from 0.1 nM to 10 mM, such as 10 nM, such as 25 nM such as 100 nM such as 250 nM.

20. The respirable aqueous pharmaceutical composition according to any one of claims 1 to 19, wherein the aqueous composition is made into an aerosol using a jet nebulizer or a mesh nebulizer.

21. The respirable aqueous pharmaceutical composition according to any one of claims 1 to 20, wherein the respirable aqueous composition is in aerosol form having a droplet size, defined as its median aerodynamic diameter (MMAD), of between 0.5 to 10 μm, such as between 0.5 to 9 μm, 0.5 to 8 μm, 0.5 to 7 μm, 0.5 to 6 μm or 0.5 to 5 μm, preferably between 0.5 to 5 μm, whereby the composition may enter the alveoli of the lung.

22. A reservoir for a nebulizer, the reservoir comprising the respirable aqueous pharmaceutical composition according to any one of claims 1 to 21.

23. A nebulizer with a reservoir comprising the reparable pharmaceutical composition according to any one of claims 1 to 21.

24. The nebulizer according to claim 23, wherein the nebulizer is a jet nebulizer or a mesh nebulizer

25. The respirable aqueous pharmaceutical composition according to any one of claims 1 to 21, for use in neutralizing active virus particles binding to human Angiotensin-converting enzyme 2 (SEQ ID NO 1), such as Severe acute respiratory syndrome coronavirus 2 (SARS-CoV-2), severe acute respiratory syndrome coronavirus (SARS-CoV) or Human coronavirus NL63 (HCoV-NL63) virus particles.

26. The respirable aqueous pharmaceutical composition according to any one of claims 1 to 21, for use for use in reducing the number of active virus particles being exhaled by subject infected by a virus binding to ACE2, such as SARS-CoV-2, SARS-CoV or HCoV-NL63.

27. The respirable aqueous pharmaceutical composition according to any one of claims 1 to 21, for use in reducing the number of active virus particles in the airways and lungs of a subject infected by a virus binding to ACE2, such as SARS-CoV-2, SARS-CoV or HCoV-NL6, whereby the inactive virus particles slow down disease progression while enabling the subject to develop natural immunity to the virus binding to human Angiotensin-converting enzyme 2 (ACE2).

28. The respirable aqueous pharmaceutical composition according to any one of claims 1 to 21, for use in the treatment or the prophylactic treatment of a patient being infected with a virus binding to human Angiotensin-converting enzyme 2 (ACE2); preferably said virus being selected from the group consisting of SARS-CoV-2, SARS-CoV, and HCoV-NL63.

29. The respirable aqueous pharmaceutical composition for use according to claim 26, wherein the respirable aqueous pharmaceutical composition is administered to the lung of the patient.

30. The respirable aqueous pharmaceutical composition for use according to claim 26 or 27, wherein said respirable aqueous pharmaceutical composition is administered to the patient as an aerosol.
